(19) United States
(12) Patent Application Publication
(10) Pub. No.: US 2009/0047703 A1
(43) Pub. Date: Feb. 19, 2009

(54) ERG-1 Peptides and Polynucleotides and Their Use in the Treatment and Diagnosis of Disease

(75) Inventor: Matthew C. Trudeau, Baltimore, MD (US)

Correspondence Address: EDELL SHAPIRO & FINNAN , LLC, 1901 RESEARCH BLVD, SUITE 400, ROCKVILLE, MD, 20850, US

(73) Assignee: UNIVERSITY OF MARYLAND, BALTIMORE, Baltimore, MD (US)

(21) Appl. No.: 12/190,973

(22) Filed: Aug. 13, 2008

Related U.S. Application Data
(60) Provisional application No. 60/956,393, filed on Aug. 17, 2007.

(51) Int. Cl. C12Q 1/02; C12N 5/06

(52) U.S. Cl. 435/29; 435/375

(57) ABSTRACT

The present invention relates to peptide and polynucleotide fragments of ERG-1, and in particular, human ERG-1a (HERG-1a) and its isoforms, and their use in the treatment and diagnosis of disease, especially cardiac diseases, such as arrhythmias, and cancer.

### CROSS-REFERENCE TO RELATED APPLICATIONS

This application claims priority to U.S. Patent Application Ser. No. 60/956,393, filed on Aug. 17, 2007 (pending), which application is herein incorporated by reference in its entirety.

### FIELD OF THE INVENTION

The present invention relates to peptide and polynucleotide fragments of ERG-1, and in particular, human ERG-1a (HERG-1a) and its isoforms, and their use in the treatment and diagnosis of disease, especially cardiac diseases, such as arrhythmias, and cancer.

### BACKGROUND OF THE INVENTION

The mammalian heart is a rhythmic electromechanical pump whose proper function depends on the generation and propagation of action potentials (“spikes” of electrical discharge) followed by periods of relaxation and refractoriness (Nerbonne, J. M et al. (2005) “Molecular Physiology of Cardiac Repolarization,” Physiol. Rev. 85:1205-1253; Roepke, T. K. (2006) “Pharmacogenetics And Cardiac Ion Channels,” Vasc. Pharmacol. 44:90-106). The generation of myocardial action potentials reflects the sequential activation and inactivation of ion channels that conduct depolarizing, inward (Na⁺ and Ca²⁺), and repolarizing, outward (K+), currents (Antzelevitch, C. et al. (2002) “Electrical heterogeneity in the heart: physiological, pharmacological and clinical implications,” In: HANDBOOK OF PHYSIOLOGY. THE CARDIOVASCULAR SYSTEM. THE HEART; Am. Physiol. Soc. Sect. 2, vol. I, p. 654-692; Nerbonne, J. M. et al. (2003) “Physiology and molecular biology of ion channels contributing to ventricular repolarization,” In: CONTEMPORARY CARDIOLOGY: CARDIAC REPOLARIZATION: BRIDGING BASIC AND CLINICAL SCIENCE, Gussak, I. et al. (eds.) Totowa, N J: Humana, p. 25-62).

Voltage-gated K⁺ (Kv) channels are the primary determinants of action potential repolarization in the mammalian myocardium. Two broad classes of repolarizing cardiac Kv currents have been identified: the transient, outward K⁺ currents (Iₜₒ) and the delayed, outwardly rectifying K⁺ currents (I_{K}) (Barry, D. M. et al. (1996) “Myocardial Potassium Channels: Electrophysiological And Molecular Diversity,” Annu. Rev. Physiol. 58:363-394; Nerbonne, J. M. et al. (2003) “Physiology And Molecular Biology Of Ion Channels Contributing To Ventricular Repolarization,” In: CONTEMPORARY CARDIOLOGY: CARDIAC REPOLARIZATION: BRIDGING BASIC ANDCLINICAL SCIENCE, Gussak, I. et al. (eds.) Totowa, N J: Humana, p. 25-62). Multiple forms of the transient K⁺ currents (I_{to fast}(I_{to,f}), I_{to slow} (I_{to,s}) and of the delayed rectifying K⁺ currents: I_{K(rapid)} (“I_{Kr}”), I_{K(slow)} (“I_{Ks}”) and I_{K(ultrarapid)} (“I_{Kur}”) have been identified. The unique time- and voltage-dependent properties of I_{Kr} and I_{Ks} suggest that these currents play prominent roles in action potential repolarization (Nerbonne, J. M et al. (2005) “Molecular Physiology of Cardiac Repolarization,” Physiol. Rev. 85:1205-1253).

The myocardial ion channels responsible for such currents are formed from transmembrane proteins. Changes in the properties or the functional expression of these proteins can lead to changes in action potential waveforms, synchronization, and/or propagation, and are associated with potentially life-threatening arrhythmias (Jeanne M. Nerbonne, J. M et al. (2005) “Molecular Physiology of Cardiac Repolarization,” Physiol. Rev. 85:1205-1253; Akar, F. G. et al. (2003) “Transmural Electrophysiological Heterogeneities Underlying Arrhythmogenesis In Heart Failure,” Circ. Res. 93:638-645; Akar, F. G. et al. (2000) “Cellular Basis For Dispersion Of Repolarization Underlying Reentrant Arrhythmias,” J. Electrocardiol. 33: 23-31; Antzelevitch, C. et al. (2002) “Electrical Heterogeneity In The Heart: Physiological, Pharmacological And Clinical Implications,” In: HANDBOOK OF PHYSIOLOGY. THE CARDIOVASCULAR SYSTEM. THE HEART; Am. Physiol. Soc. Sect. 2, vol. I, p. 654-692; Kléber, A. G. et al. (2004) “Basic Mechanisms Of Cardiac Impulse Propagation And Associated Arrhythmias,” Physiol. Rev. 84:431-488). Accordingly, the molecular, cellular, and systemic mechanisms through which such proteins contribute to the generation and maintenance of normal cardiac rhythm is of fundamental importance in the diagnosis and therapy of cardiac dysfunction.

The ERG (eag-related gene) proteins are a family of transmembrane proteins found in mammals. Three human ERG proteins (HERG-1, HERG-2,and HERG-3) have been identified. HERG-1 (also known as KCNH2) is expressed in neural and smooth muscle tissues, but is most highly expressed in the heart (Warmke, J. W. et al. (1994) “A Family Of Potassium Channel Genes Related To Eag In Drosophila And Mammals,” Proc. Natl. Acad. Sci. (USA) 91(8):3438-3442; Trudeau, M. C. et al. (1995) “HERG, A Human Inward Rectifier In The Voltage- Gated Potassium Channel Family,” Science 269:92-95; Shi, W. et al. (1997) “Identification Of Two Nervous System-Specific Members Of The Erg Potassium Channel Gene Family,” J. Neurosci. 17:9423-9432). Two HERG-1 proteins have been identified: HERG-1a and HERG-1b (London, B. et al. (1997) “Two Isoforms Of The Mouse Ether-A-Go-Go-Related Gene Coassemble To Form Channels With Properties Similar To The Rapidly Activating Component Of The Cardiac Delayed Rectifier K+ Current,” Circ. Res. 81(5):870-878; Lees-Miller, J. P. et al. (1997) “Electrophysiological Characterization Of An Alternatively Processed ERG K+ Channel In Mouse And Human Hearts,” Circ. Res. 81(5):719-726.). The HERG-1a protein has been found to assemble as tetrameric complexes that form the channels which conduct the I_{Kr} (Warmke, J. W. et al. (1994) “A Family Of Potassium Channel Genes Related To Eag In Drosophila And Mammals,” Proc. Natl. Acad. Sci. (USA) 91(8):3438-3442; Trudeau, M. C. et al. (1995) “HERG, A Human Inward Rectifier In The Voltage-Gated Potassium Channel Family,” Science 269:92-95; Delmar M. (1992) “Role Of Potassium Currents On Cell Excitability In Cardiac Ventricular Myocytes,” J. Cardiovasc. Electrophysiol 3:474-486; Sanguinetti, M. C. et al. (1995) “A Mechanistic Link Between An Inherited And An Acquired Cardiac Arrhythmia: HERG Encodes The I_{Kr} Potassium Channel,” Cell 81(2):299-307; Tseng, G. N. (2001) “I_{(Kr)}: the hERG Channel,” J. Mol. Cell. Cardiol. 33(5):835-849). HERG-1b is a cardiac-specific splice form of HERG-1a; it possesses a divergent N-terminal region and lacks the extreme N-terminal region and Pas domain. HERG-2and HERG-3 (U.S. Pat. Nos. 6,087,488and 5,986,081) are expressed exclusively in the nervous system (Shi, W. et al. (1997) “Identification Of Two Nervous System-Specific Members Of The Erg Potassium Channel Gene Family,” J. Neurosci. 17:9423-9432).

HERG channels (including their cardiac muscle counterparts, the I_{Kr} channels) mediate inward rectification because they also inactivate very rapidly at positive potentials (Smith, P. L. (1996) “The Inward Rectification Mechanism Of The HERG Cardiac Potassium Channel,” Nature 379:833-836; Vaz, R. J. et al. (2005) “Human Ether-A-Go-Go Related Gene (HERG): A Chemist's Perspective,” Prog. Med. Chem. 43:1-18; Vandenberg, J. I. et al. (2004) “The HERG K⁺ Channel: Progress In Understanding The Molecular Basis Of Its Unusual Gating Kinetics,” Eur. Biophys. J. 33(2):89-97). Then, as membrane repolarization occurs, HERG channels recover rapidly from inactivation and pass large currents before they close, thereby accomplishing the inward rectification (Roepke, T. K. (2006) “Pharmacogenetics And Cardiac Ion Channels,” Vasc. Pharmacol. 44:90-106; Tseng, G. N. (2001) “I_{(Kr)}: the hERG Channel,” J. Mol. Cell. Cardiol. 33(5):835-849). These properties ensure that HERG channels supply a strong repolarizing force towards the end of the ventricular action potential.

A central characteristic of HERG channels is their slow deactivation kinetics (Sanguinetti, M. C. et al. (1995) “A Mechanistic Link Between An Inherited And An Acquired Cardiac Arrhythmia: HERG Encodes The I_{Kr} Potassium Channel,” Cell 81(2):299-307). On depolarization, a change in membrane potential from about −80 mV to +10 mV, HERG channels open transiently and then rapidly inactivate to a non-conducting state. On repolarization, when the membrane potential returns to the ‘resting’ value of −80 mV, HERG channels have to pass back through the open (conducting) state before closing. The open-to-closed transition, referred to as deactivation, is relatively slow. The slow deactivation kinetics determine the length of action potentials involving HERG and thus control cardiac excitability (Sansom, M. S. P. (1999) “Ion channels: Structure Of A Molecular Brake,” Curr Biol. 9(5):R173-5). The slow deactivation kinetics of HERG and I_{Kr} channels are significant as they shape the “resurgent current” which drives the terminal repolarization phase of the ventricular action potential.

Mutations in HERG-1a have been found to be associated with numerous disorders including: “long QT syndrome,” a genetic disorder of cardiac repolarization that predisposes affected individuals to arrhythmia and sudden death (e.g., Sudden Infant Death Syndrome (SIDS) (U.S. Pat. Nos. 6,207,383 and 5,599,673; Sanchez-Chapula, J. A. et al. (2002) “Molecular Determinants Of Voltage-Dependent Human Ether-A-Go-Go Related Gene (HERG) K⁺ Channel Block,” J. Biol. Chem. 277(26):23587-23595; el-Sherif, N. et al. (1996) “The Electrophysiological Mechanism Of Ventricular Arrhythmias In The Long QT Syndrome. Tridimensional Mapping Of Activation And Recovery Patterns,” Circ. Res. 79(3):474-492; Thomas, D. et al. (2006) “The Cardiac Herg/Ikr Potassium Channel As Pharmacological Target: Structure, Function, Regulation, And Clinical Applications,” Curr Pharm Des. 12(18):2271-2283); “short QT syndrome,” a congenital disease associated with familial atrial fibrillation and/or sudden death or syncope (Borggrefe, M. et al. (2005) “Short QT syndrome Genotype-phenotype correlations,” J. Electrocardiol. 38(4 Suppl):75-80; Schimpf, R. et al. (2005) “Short QT Syndrome,” Cardiovasc Res. 67(3):357-366); and cancer (Camacho, J. (2006) “Ether A Go-Go Potassium Channels And Cancer,” Cancer Letters 233:1-9; Pillozzi, S. et al. (2002) “HERG Potassium Channels Are Constitutively Expressed In Primary Human Acute Myeloid Leukemias And Regulate Cell Proliferation Of Normal And Leukemic Hemopoietic Progenitors,” Leukemia 16:1791-1798; Smith, G. A. M. et al. (2002) “Functional Up-Regulation Of HERG KC Channels In Neoplastic Hematopoietic Cells,” J. Biol. Chem. 277:18528-18534; Suzuki, T. et al. (2004) “Selective Expression Of HERG And Kv2 Channels Influences Proliferation Of Uterine Cancer Cells,” Int. J. Oncol. 25:153-159).

Since HERG channel deactivation gating is essential for the normal cardiac action potential and heart rhythm, an improved understanding of the gating, stoichiometry or function of cardiac I_{Kr} channels would provide improved means for diagnosing and treating HERG-1-associated disorders. The present application is directed to these and related needs.

### SUMMARY OF THE INVENTION

The present invention relates to peptide and polynucleotide fragments of ERG-1, and in particular, human ERG-1a (HERG-1a) and its isoforms, and their use in the treatment and diagnosis of disease, especially cardiac diseases, such as arrhythmias, and cancer.

The ether á go-go-related gene (ERG) encodes proteins that form K⁺ channels in the heart which play a critical role in cardiac excitability. The human homolog of this gene is HERG-1a or KCNH2). HERG-1a and a cardiac-specific splice variant, HERG-1b, form K⁺ channels that are the central, pore-forming subunits of the rapid component of the delayed-rectifier K⁺ current′ (I_{Kr}) in heart. Cardiac I_{Kr} channels help to repolarize heart cells by conducting an outward K⁺ current during the late phases of the cardiac action potential. HERG-1a and HERG-1b subunits have several specializations that perfectly suit them for their key role in repolarization. In particular, the closing rate (deactivation) of HERG channels is a key determinant of the peak outward K⁺ current. Deactivation in HERG-1a channels is modulated by the N-terminal region of the protein. Within this region, a key determinant of deactivation is a Per-Arnt-Sim(PAS) domain. HERG-1b channels have a divergent N-terminal region that does not contain a PAS domain. Consistent with a modulatory role for the PAS domain, deactivation kinetics in HERG-1b channels are approximately 10-fold faster than that in HERG1 a channels. The present invention relates to peptide and polypeptide fragments of ERG-1a and ERG-1b (and in particular, HERG-1a and HERG-1b) that enable restoration of ERG channel function and provide means to assay ERG channels. Such assays are useful in diagnosing ERG channel dysfunction, and in the isolation of ERG channel effectors.

The present invention thus relates to the elucidation of the fundamental molecular mechanisms that underlie the deactivation gating (closing) in HERG-1a K⁺ channels and the counterpart I_{Kr} channels in cardiac muscle, and the exploitation of such mechanisms to provide improved means for diagnosing and treating HERG-1-associated disorders. The invention derives in part from the recognition that: (1) the slow deactivation gating of HERG channels is caused through electrostatic interactions between specific charged residues in the PAS-CAP region and charged residues in an intracellular region near the channel voltage-sensor domain; (2) the PAS domain:PAS receptor site interaction is formed by specific hydrophobic interactions between amino acid residues at the surface of the PAS domain and hydrophobic residues located at intracellular sites in the HERG channel; and (3) the differences in deactivation gating kinetics between HERG-1 channels and cardiac I_{Kr} channels are due to the function of HERG-1b subunits.

In detail, the invention provides a method for decreasing the deactivation kinetics of the I_{Kr} current of a mammalian cardiac cell which comprises providing to the cell a compound that specifically antagonizes a function of ERG-1b or a function of an ERG-1a molecule that comprises a mutation relative to the amino acid sequence of the wild-type ERG1a protein.

The invention particularly concerns the embodiments of such method wherein the cell is a human cell, the ERG-1b is HERG-1b, the ERG-1a is HERG-1a and/or wherein the compound comprises a polypeptide or peptide fragment of the Amino Terminal Domain of HERG-1a, or wherein the compound is the Amino Terminal Domain of HERG-1a.

The invention also provides a method for increasing the deactivation kinetics of the I_{Kr} current of a mammalian cardiac cell which comprises providing to the cell a compound that specifically antagonizes a function of ERG-1a.

The invention further concerns the embodiments of such method wherein the cell is a human cell, and the ERG-1a is HERG-1a, and/or wherein the compound comprises a polypeptide or peptide fragment of HERG-1b or of HERG-1a isoform 3 or 4.

The invention further concerns the embodiments of such methods wherein the provision of the compound to the cell is accomplished by providing to the cell a polynucleotide encoding the compound under conditions sufficient to cause expression of the polynucleotide.

The invention further concerns the embodiments of such methods wherein the mammal suffers from a condition selected from the group consisting of a hereditary Long QT Syndrome and an acquired Long QT Syndrome, and the method comprises a therapy for the condition.

The invention also provides a method for evaluating ERG channel composition or function in a sample membrane containing the channel, wherein the method comprises the steps of:

(A) providing to the sample membrane a compound that specifically antagonizes a function of an ERG-1 subunit of an ERG channel; and

(B) determining the effect of the compound on the deactivation kinetics of the I_{Kr} current of the sample membrane relative to the deactivation kinetics of the I_{Kr} current of a reference membrane in the presence of the compound;

wherein a difference in the effect of the compound on the I_{Kr} current deactivation kinetics of the sample membrane relative to the reference membrane indicates that the sample membrane exhibits abnormal ERG channel composition or function.

The invention also provides a method for determining whether an agent affects ERG channel function, wherein the method comprises the steps of:

(A) providing the agent to a membrane that comprises an ERG channel; and

(B) determining whether the agent alters the deactivation kinetics of the I_{Kr} current of the membrane;

wherein a difference in the I_{Kr} current deactivation kinetics of the membrane in the presence of the agent relative to the I_{Kr} current deactivation kinetics of the membrane in the absence of the agent indicates that the agent affects ERG channel function.

The invention further concerns the embodiment of such methods wherein the sample membrane is the membrane of a cell or wherein the sample membrane is an in vitro membrane. The invention further concerns the embodiment of such methods wherein the ERG-1 subunit is ERG-1a, and/or wherein the compound is ERG-b1. The invention further concerns the embodiment of such methods wherein the ERG-1 subunit is ERG-1b, and/or wherein the compound comprises a polypeptide or peptide fragment of the Amino Terminal Domain of HERG-1a.

The invention further concerns the embodiment of all such methods wherein the agent is an antiarrhythmic agent, a non-antiarrhythmic agent, or an antineoplastic agent.

### BRIEF DESCRIPTION OF THE FIGURES

FIG. 1 shows a schematic representation of the domain structure of the HERG-1a protein. Transmembrane domains (S1-S6) are shown as cylinders.

FIG. 2 shows a two-electrode voltage clamp recording of Xenopus oocytes expressing the HERG subfamily channels: KCNH1 (top), KCNH3 (middle) and HERG-1a (KCNH2). Resurgent current is the large current at −60 mV. Slow deactivation is depicted by the underlined arrow for O to C transition.

FIG. 3 shows resurgent HERG current (point b, bottom) in response to a ventricular action potential voltage pulse (top).

FIG. 4 shows a schematic for HERG gating. Two (of the four) channel subunits are shown. The PAS domain is depicted as a solid oval and the PAS-CAP region as an open oval. The voltage-sensor (S3-S4 paddle region) is labeled with + charges. Channels change conformation with depolarization (left to right) and recover to the closed state with repolarization (right to left). The transition from the OpenN state to the Open state (short arrow) depicts slow deactivation.

FIG. 5 shows a schematic representation of the domain structure of the HERG-1b protein. The novel N-terminal region is depicted.

FIG. 6 shows various mutations in HERG-1a that result in an increase in deactivation kinetics of K⁺ channels in the heart, which lead to altered excitability in the heart, including long QT sydromes.

FIG. 7 shows recovery (rescue) of slow deactivation by the HERG-1a N-terminal domain. Two-electrode voltage-clamp recordings of current traces from oocytes expressing HERG-1a-Citrine (Panel A); HERG N_{Del}-Citrine (Panel B); and HERG-1a N_{Del} plus the HERG-1a N-Terminal Domain fused to fused to eCFP (cyan fluorescent protein (Panel C). Slow deactivation of the current (Panel C, arrow) was restored. Also shown are representations of confocal images from Xenopus oocytes expressing the constructs. Excitation was 458 nm for eCFP and 514 nm for Citrine.

FIG. 8 shows two-electrode voltage-clamp recordings of current traces from oocytes expressing HERG-1a N_{Del} Citrine+soluble Cerulean fluorescent protein (an eCFP derivative (Panel B); HERG-1a N_{Del} plus HERG-1a N Terminal Domain Variant (F29L) (Panel C); HERG-1a N_{Del} plus HERG-1a N Terminal Domain Variant (Y43A) (Panel D); Panel A shows a water control. Scale bar is 1 μA and 0.2 s. Also shown are representations of confocal images from Xenopus oocytes expressing the constructs. Excitation was 458 nm for eCFP and 514 nm for Citrine.

FIG. 9 shows a Box plot of time constants for deactivation at −100 mV.

FIG. 10 shows electrode voltage clamp recordings of currents from HERG channels with a mutation in the PAS domain at position 43 (Panel A) and 31 (Panel B) with and without addition of the HERG-1a N-terminal domain.

FIG. 11 shows two-electrode voltage-clamp recordings from HERG-1a-Citrine (Panel A) and HERG-1a Citrine +HERG-1a N Terminal Domain-eCFP (Panel B).

FIG. 12 shows two-electrode voltage-clamp recordings from HERG-1b (Panel A) and HERG-1b +HERG-1a N Terminal Domain-eCFP (Panel B).

FIG. 13 shows two-electrode voltage-clamp recordings from Xenopus oocytes expressing HERG channels formed from co-expression of HERG1a and HERG1b (Panel A) and HERG1a, HERG1b and the HERG1a N-terminal domain (Panel B). Slower deactivation kinetics were detected for channels in Panel B (arrow). Panel C shows a Box plot of deactivation time constants (ms) from current relaxation measured at −100 mV after pulse to 20 mV as in A and B (n=4). The means are the center lines of the box plots and are significantly different (P<.001) from ANOVA.

FIG. 14 shows two-electrode voltage-clamp recordings of currents from Xenopus oocytes expressing mERG1a (Panel A, upper) and mERG1b (Panel A, lower). Inward tail current from co-expression of mERG1a and mERG1b (Panel B, thick dashed trace) after the same voltage pulse in Panel A, but shown only at −100 mV. Weighted sums of homomeric mERG1a and mERG1b (thin solid, dotted and dashed traces, B). Figure is modified from London, B. et al. (1997) (“Two Isoforms Of The Mouse Ether-A-Go-Go-Related Gene Co-Assemble To Form Channels With Properties Similar To The Rapidly Activating Component Of The Cardiac Delayed Rectifier K+ Current,” Circ. Res. 81(5):870-878)).

FIG. 15 shows spectral measurement of FRET with stimulated emission of acceptor fluorophore. Emission spectra from whole oocytes expressing HERG-1b-eCFP and HERG-1a-Citrine (Left panel) and HERG-1a-Citrine alone (Right panel). The F₄₈₈ traces (solid lines, left and right) are the emission of HERG-1a-Citrine after excitation with the 488 laser. The experimental (eCFP and Citrine) spectra (dotted, left) was obtained after excitation at 458. A scaled trace from an eCFP-only control (dashed) was subtracted from the dotted trace (left) to give the F₄₅₈ trace (thick dashed, left) which contains a FRET component and a direct component. F₄₅₈ (thick dashed, right) is the direct excitation of HERG-1a Citrine by the 458 laser.

### DESCRIPTION OF PREFERRED EMBODIMENTS OF THE INVENTION

The ether á go-go-related gene encodes proteins that form K⁺ channels in the heart which play a critical role in cardiac excitability. The human homolog of this gene is HERG-1a or KCNH2). HERG-1a and a cardiac-specific splice variant, HERG-1b, form K⁺ channels that are the central, pore-forming subunits of the rapid component of the delayed-rectifier K⁺ current′ (I_{Kr}) in heart. Cardiac I_{Kr} channels help to repolarize heart cells by conducting an outward K⁺ current during the late phases of the cardiac action potential. HERG-1a and HERG-1b subunits have several specializations that perfectly suit them for their key role in repolarization. In particular, the closing rate (deactivation) of HERG channels is a key determinant of the peak outward K⁺ current. Deactivation in HERG-1a channels is modulated by the N-terminal region of the protein. Within this region, a key determinant of deactivation is a Per-Arnt-Sim(PAS) domain. HERG-1b channels have a divergent N-terminal region that does not contain a PAS domain. Consistent with a modulatory role for the PAS domain, deactivation kinetics in HERG-1b channels are approximately 10-fold faster than that in HERGI a channels. The present invention relates to peptide and polypeptide fragments of ERG-1a and ERG-1b (and in particular, HERG-1a and HERG-1b) that enable restoration of ERG channel function and provide means to assay ERG channels. Such assays are useful in diagnosing ERG channel dysfunction, and in the isolation of ERG channel effectors.

### A. The Proteins, Polypeptides and Peptides of the Present Invention

As used herein, the term “ERG” is intended to refer to an ERG (eag-related gene) protein. The term “HERG” denotes the human ERG protein; “MERG” denotes the murine ERG protein, etc. (London, B. et al. (1997) “Two Isoforms Of The Mouse Ether-A-Go-Go-Related Gene Co-Assemble To Form Channels With Properties Similar To The Rapidly Activating Component Of The Cardiac Delayed Rectifier K⁺ Current,” Circ. Res. 81(5):870-878; Zehelein, J. et al. (2001) “Molecular Cloning And Expression Of Cerg, The Ether A Go-Go-Related Gene From Canine Myocardium,” Pflugers Arch. 442(2):188-191; Warmke, J. W. et al. (1994) “A Family Of Potassium Channel Genes Related To Eag In Drosophila And Mammals,” Proc. Natl. Acad. Sci. (USA) 91(8):3438-3442). The invention is illustrated with respect to human ERG proteins and their uses, but is intended to encompass other ERG proteins and their respective uses as well.

The term “ERG-1a” is intended to denote the ERG protein responsible for the I_{Kr} ptassium channel discussed above. The term “HERG-1a” is intended to denote the HERG-1 isoform having 1159 amino acid residues in length having the following sequence (GenBank U04246; SEQ ID NO:1):

MPVRRGHVAP QNTFLDTIIR KFEGQSRKFI TANARVENCA
VIYCNDGFCE LCGYSRAEVM QRPCTCDFLH GPRTQRRAAA
QIAQALLGAE ERKVEIAFYR KDGSCFLCLV DVVPVKNEDG
AVIMFILNFE VVMEKDMVGS PAHDTNHRGP PTSWLAPGRA
KTFRLKLPAL LALTARESSV RSGGAGGAGA PGAVVVDVDL
TPAAPSSESL ALDEVTAMDN HVAGLGPAEE RRALVGPGSP
PRSAPGQLPS PRAHSLNPDA SGSSCSLART RSRESCASVR
RASSADDIEA MRAGVLPPPP RHASTGAMHP LRSGLLNSTS
DSDLVRYRTI SKIPQITLNF VDLKGDPFLA SPTSDREIIA
PKIKERTHNV TEKVTQVLSL GADVLPEYKL QAPRIHRWTI
LHYSPFKAVW DWLILLLVIY TAVFTPYSAA FLLKETEEGP
PATECGYACQ PLAVVDLIVD IMFIVDILIN FRTTYVNANE
EVVSHPGRIA VHYFKGWFLI DMVAAIPFDL LIFGSGSEEL
IGLLKTARLL RLVRVARKLD RYSEYGAAVL FLLMCTFALI
AHWLACIWYA IGNMEQPHMD SRIGWLHNLG DQIGKPYNSS
GLGGPSIKDK YVTALYFTFS SLTSVGFGNV SPNTNSEKIF
SICVMLIGSL MYASIFGNVS AIIQRLYSGT ARYHTQMLRV
REFIRFHQIP NPLRQRLEEY FQHAWSYTNG IDMNAVLKGF
PECLQADICL HLNRSLLQHC KPFRGATKGC LRALAMKFKT
THAPPGDTLV HAGDLLTALY FISRGSIEIL RGDVVVAILG
KNDIFGEPLN LYARPGKSNG DVRALTYCDL HKIHRDDLLE
VLDMYPEFSD HFWSSLEITF NLRDTNMIPG SPGSTELEGG
FSRQRKRKLS FRRRTDKDTE QPGEVSALGP GRAGAGPSSR
GRPGGPWGES PSSGPSSPES SEDEGPGRSS SPLRLVPFSS
PRPPGEPPGG EPLMEDCEKS SDTCNPLSGA FSGVSNIFSF
WGDSRGRQYQ ELPRCPAPTP SLLNIPLSSP GRRPRGDVES
RLDALQRQLN RLETRLSADM ATVLQLLQRQ MTLVPPAYSA
VTTPGPGPTS TSPLLPVSPL PTLTLDSLSQ VSQFMACEEL
PEGAPELEQE GPTRRLSLPG QLGALTSQPL HRHGSDPGS

HERG-1a is encoded by a polynucleotide having the sequence (SEQ ID NO:2):

ATGCCGGTGC GGAGGGGCCA CGTCGCGCCG CAGAACACCT
TCCTGGACAC CATCATCCGC AAGTTTGAGG GCCAGAGCCG
TAAGTTCATC ATCGCCAACG CTCGGGTGGA GAACTGCGCC
GTCATCTACT GCAACGACGG CTTCTGCGAG CTGTGCGGCT
ACTCGCGGGC CGAGGTGATG CAGCGACCCT GCACCTGCGA
CTTCCTGCAC GGGCCGCGCA CGCAGCGCCG CGCTGCCGCG
CAGATCGCGC AGGCACTGCT GGGCGCCGAG GAGCGCAAAG
TGGAAATCGC CTTCTACCGG AAAGATGGGA GCTGCTTCCT
ATGTCTGGTG GATGTGGTGC CCGTGAAGAA CGAGGATGGG
GCTGTCATCA TGTTCATCCT CAATTTCGAG GTGGTGATGG
AGAAGGACAT GGTGGGGTCC CCGGCTCATG ACACCAACCA
CCGGGGCCCC CCCACCAGCT GGCTGGCCCC AGGCCGCGCC
AAGACCTTCC GCCTGAAGCT GCCCGCGCTG CTGGCGCTGA
CGGCCCGGGA GTCGTCGGTG CGGTCGGGCG GCGCGGGCGG
CGCGGGCGCC CCGGGGGCCG TGGTGGTGGA CGTGGACCTG
ACGCCCGCGG CACCCAGCAG CGAGTCGCTG GCCCTGGACG
AAGTGACAGC CATGGACAAC CACGTGGCAG GGCTCGGGCC
CGCGGAGGAG CGGCGTGCGC TGGTGGGTCC CGGCTCTCCG
CCCCGCAGCG CGCCCGGCCA GCTCCCATCG CCCCGGGCGC
ACAGCCTCAA CCCCGACGCC TCGGGCTCCA GCTGCAGCCT
GGCCCGGACG CGCTCCCGAG AAAGCTGCGC CAGCGTGCGC
CGCGCCTCGT CGGCCGACGA CATCGAGGCC ATGCGCGCCG
GGGTGCTGCC CCCGCCACCG CGCCACGCCA GCACCGGGGC
CATGCACCCA CTGCGCAGCG GCTTGCTCAA CTCCACCTCG
GACTCCGACC TCGTGCGCTA CCGCACCATT AGCAAGATTC
CCCAAATCAC CCTCAACTTT GTGGACCTCA AGGGCGACCC
CTTCTTGGCT TCGCCCACCA GTGACCGTGA GATCATAGCA
CCTAAGATAA AGGAGCGAAC CCACAATGTC ACTGAGAAGG
TCACCCAGGT CCTGTCCCTG GGCGCCGACG TGCTGCCTGA
GTACAAGCTG CAGGCACCGC GCATCCACCG CTGGACCATC
CTGCATTACA GCCCCTTCAA GGCCGTGTGG GACTGGCTCA
TCCTGCTGCT GGTCATCTAC ACGGCTGTCT TCACACCCTA
CTCGGCTGCC TTCCTGCTGA AGGAGACGGA AGAAGGCCCG
CCTGCTACCG AGTGTGGCTA CGCCTGCCAG CCGCTGGCTG
TGGTGGACCT CATCGTGGAC ATCATGTTCA TTGTGGACAT
CCTCATCAAC TTCCGCACCA CCTACGTCAA TGCCAACGAG
GAGGTGGTCA GCCACCCCGG CCGCATCGCC GTCCACTACT
TCAAGGGCTG GTTCCTCATC GACATGGTGG CCGCCATCCC
CTTCGACCTG CTCATCTTCG GCTCTGGCTC TGAGGAGCTG
ATCGGGCTGC TGAAGACTGC GCGGCTGCTG CGGCTGGTGC
GCGTGGCGCG GAAGCTGGAT CGCTACTCAG AGTACGGCGC
GGCCGTGCTG TTCTTGCTCA TGTGCACCTT TGCGCTCATC
GCGCACTGGC TAGCCTGCAT CTGGTACGCC ATCGGCAACA
TGGAGCAGCC ACACATGGAC TCACGCATCG GCTGGCTGCA
CAACCTGGGC GACCAGATAG GCAAACCCTA CAACAGCAGC
GGCCTGGGCG GCCCCTCCAT CAAGGACAAG TATGTGACGG
CGCTCTACTT CACCTTCAGC AGCCTCACCA GTGTGGGCTT
CGGCAACGTC TCTCCCAACA CCAACTCAGA GAAGATCTTC
TCCATCTGCG TCATGCTCAT TGGCTCCCTC ATGTATGCTA
GCATCTTCGG CAACGTGTCG GCCATCATCC AGCGGCTGTA
CTCGGGCACA GCCCGCTACC ACACACAGAT GCTGCGGGTG
CGGGAGTTCA TCCGCTTCCA CCAGATCCCC AATCCCCTGC
GCCAGCGCCT CGAGGAGTAC TTCCAGCACG CCTGGTCCTA
CACCAACGGC ATCGACATGA ACGCGGTGCT GAAGGGCTTC
CCTGAGTGCC TGCAGGCTGA CATCTGCCTG CACCTGAACC
GCTCACTGCT GCAGCACTGC AAACCCTTCC GAGGGGCCAC
CAAGGGCTGC CTTCGGGCCC TGGCCATGAA GTTCAAGACC
ACACATGCAC CGCCAGGGGA CACACTGGTG CATGCTGGGG
ACCTGCTCAC CGCCCTGTAC TTCATCTCCC GGGGCTCCAT
CGAGATCCTG CGGGGCGACG TCGTCGTGGC CATCCTGGGG
AAGAATGACA TCTTTGGGGA GCCTCTGAAC CTGTATGCAA
GGCCTGGCAA GTCGAACGGG GATGTGCGGG CCCTCACCTA
CTGTGACCTA CACAAGATCC ATCGGGACGA CCTGCTGGAG
GTGCTGGACA TGTACCCTGA GTTCTCCGAC CACTTCTGGT
CCAGCCTGGA GATCACCTTC AACCTGCGAG ATACCAACAT
GATCCCGGGC TCCCCCGGCA GTACGGAGTT AGAGGGTGGC
TTCAGTCGGC AACGCAAGCG CAAGTTGTCC TTCCGCAGGC
GCACGGACAA GGACACGGAG CAGCCAGGGG AGGTGTCGGC
CTTGGGGCCG GGCCGGGCGG GGGCAGGGCC GAGTAGCCGG
GGCCGGCCGG GGGGGCCGTG GGGGGAGAGC CCGTCCAGTG
GCCCCTCCAG CCCTGAGAGC AGTGAGGATG AGGGCCCAGG
CCGCAGCTCC AGCCCCCTCC GCCTGGTGCC CTTCTCCAGC
CCCAGGCCCC CCGGAGAGCC GCCGGGTGGG GAGCCCCTGA
TGGAGGACTG CGAGAAGAGC AGCGACACTT GCAACCCCCT
GTCAGGCGCC TTCTCAGGAG TGTCCAACAT TTTCAGCTTC
TGGGGGGACA GTCGGGGCCG CCAGTACCAG GAGCTCCCTC
GATGCCCCGC CCCCACCCCC AGCCTCCTCA ACATCCCCCT
CTCCAGCCCG GGTCGGCGGC CCCGGGGCGA CGTGGAGAGC
AGGCTGGATG CCCTCCAGCG CCAGCTCAAC AGGCTGGAGA
CCCGGCTGAG TGCAGACATG GCCACTGTCC TGCAGCTGCT
ACAGAGGCAG ATGACGCTGG TCCCGCCCGC CTACAGTGCT
GTGACCACCC CGGGGCCTGG CCCCACTTCC ACATCCCCGC
TGTTGCCCGT CAGCCCCCTC CCCACCCTCA CCTTGGACTC
GCTTTCTCAG GTTTCCCAGT TCATGGCGTG TGAGGAGCTG
CCCCCGGGGG CCCCAGAGCT TCCCCAAGAA GGCCCCACAC
GACGCCTCTC CCTACCGGGC CAGCTGGGGG CCCTCACCTC
CCAGCCCCTG CACAGACACG GCTCGGACCC GGGCAGTTAG

HERG-1a possesses an intracellular amino-terminal region (located at approximately residues 1-403 of SEQ ID NO:1), six a-helical transmembrane domains: S1 (located at approximately residues 404-424 of SEQ ID NO:1), S2 (located at approximately residues 451-471 of SEQ ID NO:1), S3 (located at approximately residues 496-519 of SEQ ID NO:1), S4 (located at approximately residues 521-541 of SEQ ID NO:1), S5 (located at approximately residues 548-568 of SEQ ID NO:1) and S6 (located at approximately residues 639-659 of SEQ ID NO:1) and an intracellular carboxy-terminal region (located at approximately residues 660-1159 of SEQ ID NO:1). The amino terminal region contains a Per-Arnt-Sim (“PAS”) domain (located at approximately residues 41-70 of SEQ ID NO:1) and a PAS-CAP domain (located at approximately residues 1-16 of SEQ ID NO:1) (Morais Cabral, J. H. et al. (1998) “Crystal Structure And Functional Analysis Of The HERG Potassium Channel N Terminus: A Eukaryotic PAS Domain,” Cell 95:649-655). A cyclic nucleotide binding domain is present in the carboxy-terminal region (located at approximately residues 742-842 of SEQ ID NO:1) (see, Bauer, C. K. et al. (2001) “Physiology of EAG K⁺ Channels,” J. Membr. Biol. 182:1-15). Domain S4 senses the transmembrane potential, while domains S5-S6 form the K⁺-selective pore (Sanguinetti, M. C. (2006), “HERG Potassium Channels And Cardiac Arrhythmia,” Nature 440(7083):463-469).

The present invention relates to this protein as well as to variants (i.e., proteins that differ in sequence due to alternative transcription, polymorphisms or naturally arising mutation in the encoding erg-1a gene) and homolog proteins (i.e., proteins encoded by the erg-1a gene of non-human species (e.g., MERG-1a). Examples of HERG-1a variants include the three isoforms of HERG-1a (Crociani, O. et al. (2003) “Cell Cycle-Dependent Expression Of HERG-1 and HERG-1B Isoforms In Tumor Cells,” J. Biol. Chem. 278(5):2947-2955).

As used herein, the term “HERG-1b” is intended to refer to HERG-1a isoform 2 in which amino-terminal residues 1-376 of HERG-1a have been replaced with a 36 amino acid long peptide (shown underlined below). HERG-1b is thus 819 amino acids in length and has the following sequence (SEQ ID NO:3):

MAAPAGKASR TGALRPRAQK GRVRRAVRIS SLVAQEVLSL
GADVLPEYKL QAPRIHRWTI LHYSPFKAVW DWLILLLVIY
TAVFTPYSAA FLLKETEEGP PATECGYACQ PLAVVDLIVD
IMFIVDILIN FRTTYVNANE EVVSHPGRIA VHYFKGWFLI
DMVAAIPFDL LIFGSGSEEL IGLLKTARLL RLVRVARKLD
RYSEYGAAVL FLLMCTFALI AHWLACIWYA IGNMEQPHMD
SRIGWLHNLG DQIGKPYNSS GLGGPSIKDK YVTALYFTFS
SLTSVGFGNV SPNTNSEKIF SICVMLIGSL MYASIFGNVS
AIIQRLYSGT ARYHTQMLRV REFIRFHQIP NPLRQRLEEY
FQHAWSYTNG IDMNAVLKGF PECLQADICL HLNRSLLQHC
KPFRGATKGC LRALAMKFKT THAPPGDTLV HAGDLLTALY
FISRGSIEIL RGDVVVAILG KNDIFGEPLN LYARPGKSNG
DVRALTYCDL HKIHRDDLLE VLDMYPEFSD HFWSSLEITF
NLRDTNMIPG SPGSTELEGG FSRQRKRKLS FRRRTDKDTE
QPGEVSALGP GRAGAGPSSR GRPGGPWGES PSSGPSSPES
SEDEGPGRSS SPLRLVPFSS PRPPGEPPGG EPLMEDCEKS
SDTCNPLSGA FSGVSNIFSF WGDSRGRQYQ ELPRCPAPTP
SLLNIPLSSP GRRPRGDVES RLDALQRQLN RLETRLSADM
ATVLQLLQRQ MTLVPPAYSA VTTPGPGPTS TSPLLPVSPL
PTLTLDSLSQ VSQFMACEEL PPGAPELPQE GPTRRLSLPG
QLGALTSQPL HRHGSDPGS

HERG-1a isoform 3 has the sequence (SEQ ID NO:4):

MPVRRGHVAP QNTFLDTIIR KFEGQSRKFI IANARVENCA
VIYCNDGFCE LCGYSRAEVM QRPCTCDFLH GPRTQRRAAA
QIAQALLGAE ERKVEIAFYR KDGSCFLCLV DVVPVKNEDG
AVIMFILNFE VVMEKDMVGS PAHDTNHRGP PTSWLAPGRA
KTFRLKLPAL LALTARESSV RSGGAGGAGA PGAVVVDVDL
TPAAPSSESL ALDEVTAMDN HVAGLGPAEE RRALVGPGSP
PRSAPGQLPS PRAHSLNPDA SGSSCSLART RSRESCASVR
RASSADDIEA MRAGVLPPPP RHASTGAMHP LRSGLLNSTS
DSDLVRYRTI SKIPQITLNF VDLKGDPFLA SPTSDREIIA
PKIKERTHNV TEKVTQVLSL GADVLPEYKL QAPRIHRWTI
LHYSPFKAVW DWLILLLVIY TAVFTPYSAA FLLKETEEGP
PATECGYACQ PLAVVDLIVD IMFIVDILIN FRTTYVNANE
EVVSHPGRIA VHYFKGWFLI DMVAAIPFDL LTFGSGSEEL
IGLLKTARLL RLVRVARKLD RYSEYGAAVL FLLMCTFALI
AHWLACIWYA IGNMEQPHMD SRIGWLHNLG DQIGKPYNSS
GLGGPSIKDK YVTALYFTFS SLTSVGFGNV SENTNSEKIF
SICVMLIGSL MYASIFGNVS AIIQRLYSGT ARYHTQMLRV
REFIRFHQIP NPLRQRLEEY FQHAWSYTNG IDMNAVLKGF
PECLQADICL HLNRSLLQHC KPFRGATKGC LRALAMKFKT
THAPPGDTLV HAGDLLTALY FISRGSIEIL RGDVVVAILG
MGWGAGTGLE MPSAASRGAS LLNMQSLGLW TWDCLQGHWA
PLIHLNSGPP SGAMERSPTW GEAAELWGSH ILLPFRIRHK
QTLFASLK

HERG-1a isoform 4 has the sequence (SEQ ID NO:5):

MPVRRGHVAP QNTFLDTIIR KFEGQSRKFI IANARVENCA
VIYCNDGFCE LCGYSRAEVM QRPCTCDFLH GPRTQRRAAA
QIAQALLGAE ERKVEIAFYR KDGSCFLCLV DVVPVKNEDG
AVIMFILNFE VDVDLTPAAP SSESLALDEV TAMDNHVAGL
GPAEERRALV GPGSPPRSAP GQLPSPRAHS LNPDASGSSC
SLARTRSRES CASVRRASSA DDIEAMRAGV LPPPPRHAST
GAMHPLRSGL LNSTSDSDLV RYRTISKIPQ ITLNFVDLKG
DPFLASPTSD REIIAPKIKE RTHNVTEKVT QVLSLGADVL
PEYKLQAPRI HRWTILHYSP FKAVWDWLIL LLVIYTAVFT
PYSAAFLLKE TEEGEPATEC GYACQPLAVV DLIVDIMFIV
DILINFRTTY VNANEEVVSH PGRIAVHYFK GWFLIDMVAA
IPFDLLIFGS GSEELIGLLK TARLLRLVRV ARKLDRYSEY
GAAVLFLLMC TFALIAHWLA CIWYAIGNME QPHMDSRIGW
LHNLGDQIGK PYNSSGLGGP SIKDKYVTAL YFTFSSLTSV
GFGNVSPNTN SEKIFSICVM LIGSLMYASI FGNVSAIIQR
LYSGTARYHT QMLRVREFIR FHQIPNPLRQ RLEEYFQHAW
SYTNGIDMNA VLKGFPECLQ ADICLHLNRS LLQHCKPFRG
ATKGCLRALA MKFKTTHAPP GDTLVHAGDL LTALYFISRG
SIEILRGDVV VAILGMGWGA GTGLEMPSAA SRGASLLNMQ
SLGLWTWDCL QGHWAPLIHL NSGPPSGAME RSPTWGEAAE
LWGSHILLPF RIRHKQTLFA SLK

Variants include HERG-1a muteins such as V198E and P202L, etc. Sequences of encompassed isoforms and variants can be found at UniProtKB/Swiss-Prot entry Q12809, herein incorporated by reference.

The invention concerns polypeptides (having a length greater than 100 amino acid residues) and peptides (having a length of less than 100 amino acid residues). The invention particularly concerns polypeptides and peptides that comprise a domain of the amino-terminal region of HERG-1a. As used herein, the term “HERG-1a NH₂-Terminal Domain” refers to residues 1-135 of the HERG-1a intracellular amino-terminal region (SEQ ID NO:6):

MPVRRGHVAP QNTFLDTIIR KFEGQSRKFI IANARVENCA
VIYCNDGFCE LCGYSRAEVM QRPCTCDFLH GPRTQRRAAA
QIAQALLGAE ERKVEIAFYR KDGSCFLCLV DVVPVKNEDG
AVIMFILNFE VVMEK

As used herein, the term “PAS-CAP” refers to residues 1-16 of the HERG-1a intracellular amino-terminal region (SEQ ID NO:7):

MPVRRGHVAP QNTFLD

As used herein, the term “compound” (including all of its forms and tenses) is a molecular entity including, for example, a small molecule (especially small organic molecules that satisfy the constraints of Lipinski's Rules (Lipinski, C. A. et al. (1997) “Experimental And Computational Approaches To Estimate Solubility And Permeability In Drug Discovery And Development Settings,” Adv. Drug Del. Rev, 23:3-25; Lipinski, C. A. et al. (2001) “Experimental And Computational Approaches To Estimate Solubility And Permeability In Drug Discovery And Development Settings,” Adv. Drug Del. Rev. 46,3-26; Oprea, T. I. et al. (2001) “Is There A Difference Between Leads And Drugs? A Historical Perspective,” J. Chem. Inf. Comput. Sci. 41:1308-1315; Arup, K. et al. (1999) “A Knowledge-Based Approach in Designing Combinatorial or Medicinal Chemistry Libraries for Drug Discovery,” J. Combin. Chem. 1:55-68), a nucleic acid (e.g., an oligonucleotide, and in particular, a siRNA, a shRNA an expression cassette, an antisense DNA, an antisense RNA, etc.), protein, peptide, antibody, antisense drug, or other biomolecule that is naturally made, synthetically made, or semi-synthetically made and is used alone or in combination with other therapies or methods for the stated purposes herein.

As used herein, the term “antagonize” (and all it forms and tenses) means to, for example, promote, facilitate, or bring about a functional change, complete or partial, of a particular protein, channel, or other functional unit of a cell. For example, antagonizing a function of an ion channel includes increasing or decreasing ion transport kinetics. In particular embodiments, antagonizing a function of ERG-1b includes decreasing the deactivation kinetics ERG-1b. In other particular embodiments, antagonizing a function of ERG-1a comprising a mutation includes decreasing the deactivation kinetics of ERG-1a comprising a mutation.

### B. HERG K⁺ Channels and Their Physiological Significance

The ERG (ether á go-go related) K⁺ channels play a highly significant role in electrical signaling, cardiac physiology and pathophysiology for several key reasons, including:
(1) ERG is a principal, pore-forming component of native cardiac I_{Kr} channels as shown by the P.I and others (Sanguinetti, M. C. et al. (1995) “A Mechanistic Link Between An Inherited And An Acquired Cardiac Arrhythmia: HERG Encodes The I_{Kr} Potassium Channel,” Cell 81(2):299-307; Trudeau, M. C. et al. (1995) “HERG, A Human Inward Rectifier In The Voltage-Gated Potassium Channel Family,” Science 269:92-95). Cardiac I_{Kr} channels play a major role in repolarizing the late phase of the action potential in heart (Sanguinetti, M. C. et al. (1990) “Two Components Of Cardiac Delayed Rectifier K⁺ Current. Differential Sensitivity To Block By Class III Antiarrhythmic Agents,” J. Gen.

Physiol. 96:195-215);
(2) Mutations in the Human ERG (HERG) gene are associated with Long QT Syndrome (Type 2), a predisposition to cardiac arrhythmias, ventricular fibrillation and sudden death (Curran, M. E. et al. (1995) “A Molecular Basis For Cardiac Arrhythmia: HERG Mutations Cause Long QT Syndrome. Cell 80:795-803. HERG mutations have also been linked to Short QT Syndrome (SQTS) and Sudden Infant Death Syndrome (SIDS); and
(3) Common pharmaceuticals, illicit drugs and dietary compounds produce an acquired syndrome similar to inherited LQTS, termed acquired LQTS (aLQTS.) The mechanistic basis of aLQTS is an inhibitory interaction of these compounds with HERG channels, which in turn reduces native cardiac I_{Kr} current (Finlayson, K. et al. (2004) “Acquired QT Interval Prolongation And HERG: Implications For Drug Discovery And Development,” Eur. J. Pharmacol. 500:129-142; Mitcheson, J. S. et al. (2000) “A Structural Basis For Drug-Induced Long QT Syndrome,” Proc. Natl. Acad. Sci. (USA) 97:12329-12333; O'Leary, M. E. (2001) “Inhibition Of Human Ether-A-Go-Go Potassium Channels By Cocaine,” Mol. Pharmacol. 59:269-77; Zhang, S. et al. (2001) “Cocaine Blocks Herg, But Not KvLQT1⁺ minK Potassium Channels,” Molec. Pharmacol. 59(5):1069-1076). Inhibition of HERG by drugs is quite prevalent and the FDA requires that all new pharmaceuticals been screened for a lack of effect on HERG channels (Roden, D. M. (2004) “Drug-Induced Prolongation of the QT Interval,” New Engl. J. Med. 350: 1013-1022).

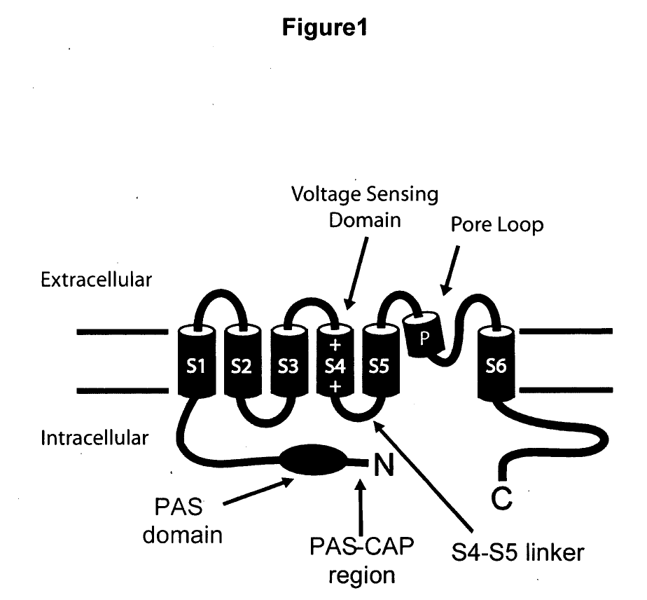

HERG channels are members of a K⁺ channel family that have six transmembrane (6TM) regions (FIG. 1) where four subunits combine to form a functional K⁺ channel around a single centrally-located pore (P) region (Warmke, J. W. et al. (1994) “A Family Of Potassium Channel Genes Related To Eag In Drosophila And Mammals,” Proc. Natl. Acad. Sci. (USA) 91(8):3438-3442). Key functional domains in HERG are a domain with homology to the Per-Arnt-Sim (PAS) domains from clock proteins (Morais Cabral, J. H. et al. (1998) “Crystal Structure And Functional Analysis Of The HERG Potassium Channel N Terminus: A Eukaryotic PAS Domain,” Cell 95:649-655) and a short region that ‘caps’ the PAS domain, the PAS-CAP region (Wang, J. et al. (2000) “Dynamic Control Of Deactivation Gating By A Soluble Amino-Terminal Domain In HERG K(⁺) Channels,” J. Gen. Physiol. 115:749-758). Like other 6TM channels, HERG channels open and close (gate) in response to changes in membrane voltage that move the S3-S4 voltage-sensor domain (Jiang, Y. et al. (2003) “X-Ray Structure Of A Voltage-Dependent K⁺ Channel,” Nature 423:33-41) and open a channel gate located in the lower S6 region (Doyle, D. A. et al. (1998) “The Structure Of The Potassium Channel: Molecular Basis Of K⁺ Conduction And Selectivity,” Science 280:69-77; Liu, Y. et al. (1997) “Gated Access To The Pore Of A Voltage-Dependent K⁺ Channel,” Neuron 19:175-184) that controls access to the pore. Like other 6TM channels, HERG appears to adhere to this basic mechanism of voltage-dependent channel opening (Hidalgo, P. et al. (1995) “Revealing The Architecture Of A K⁺ Channel Pore Through Mutant Cycles With A Peptide Inhibitor,” Science 268:307-310; Piper, D. R. et al. (2003) “Gating Currents Associated With Intramembrane Charge Displacement In HERG Potassium Channels,” Proc. Natl. Acad. Sci. (USA) 100:10534-10539; Smith, P. L. et al. (2002) “Fast And Slow Voltage Sensor Movements In HERG Potassium Channels,” J. Gen. Physiol. 119:275-293; Zhang, M. et al. (2004) “Gating Charges In The Activation And Inactivation Processes Of The HERG Channel,” J. Gen. Physiol. 124:703-718).

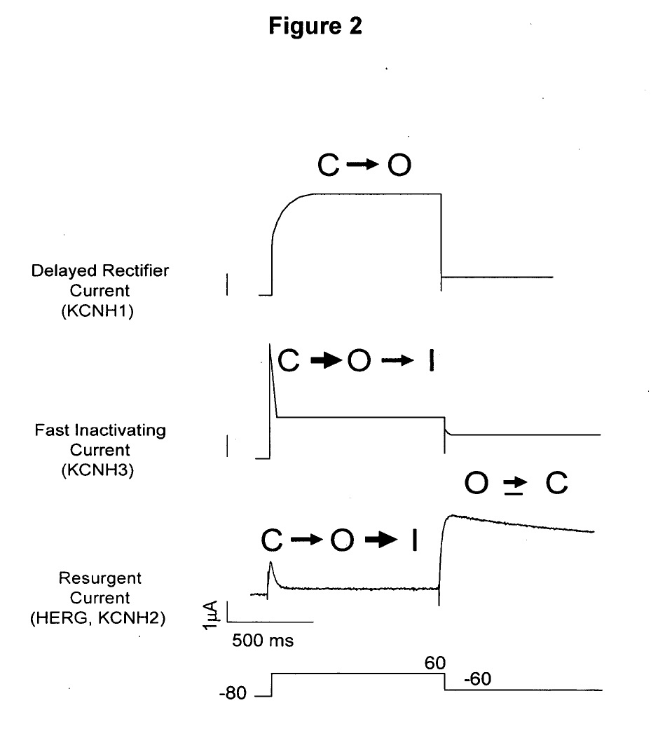

HERG channels have a distinctive response to changes in membrane voltage (Sanguinetti, M. C. et al. (1995) “A Mechanistic Link Between An Inherited And An Acquired Cardiac Arrhythmia: HERG Encodes The I_{Kr} Potassium Channel,” Cell 81(2):299-307; Trudeau, M. C. et al. (1995) “HERG, A Human Inward Rectifier In The Voltage-Gated Potassium Channel Family,” Science 269:92-95). For example, after a depolarizing pulse to 60 mV, very little outward current is detected, however upon repolarization to −60 mV a large outward, “resurgent” current (FIG. 2) is detected. The large resurgent HERG current can be explained by a common scheme used to describe conformational changes voltage-gated channels (Hille, B. (2001) “Ion Channels Of Excitable Membranes, 3rd ed. Sinauer Associates, Sunderland). Upon depolarization, HERG channels make transitions from a series of closed states (C) to an open state (O) and then quickly enter an inactive (I), non-conducting state. This accounts for the small outward current. Upon repolarization, HERG channels recover from I very quickly and re-enter O, accounting for an increase in outward current. Channels then make transitions from O to C accounting for the relaxation of the current toward zero (FIG. 2, lower) (Trudeau, M. C. et al. (1999) “Functional Analysis Of A Mouse Brain Elk-type K⁺ Channel,” J. Neurosci. 19:2906-2918; Trudeau, M. C. et al. (1995) “HERG, A Human Inward Rectifier In The Voltage-Gated Potassium Channel Family,” Science 269:92-95). Thus, the resurgent current is due to the fast recovery from inactivation (I to O transition) and the slow relaxation of current with repolarization (O to C transition). The slow relaxation of current with repolarization is slow deactivation gating (FIG. 2, underlined arrow). In contrast, delayed rectifier channels (KCNH 1, FIG. 2, upper) and channels with fast inactivation (KCNH3) (FIG. 2, middle) do not exhibit a resurgent current.

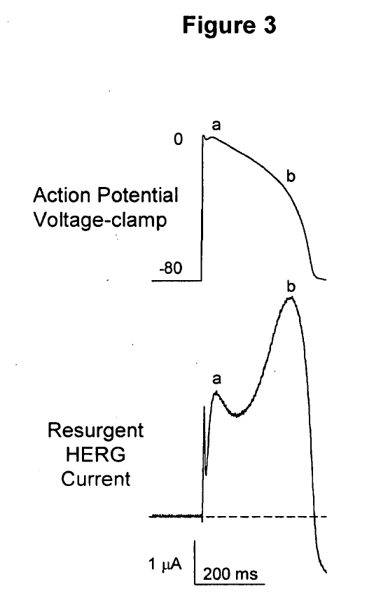

The biophysical mechanisms that underlie HERG gating, and in particular slow deactivation gating, are critical for the fundamental cardiac rhythm. In the heart, channels respond complex voltage waveforms, not voltage steps. In response to a voltage pulse that mimics a rabbit ventricular action potential, the HERG current is suppressed at an early stage since channels are in the I state (FIG. 3, a). The current peaks at the late phase of the action potential (FIG. 3, b) due to recovery from inactivation (I to O) and slow deactivation (O to C). In this way, HERG is specialized to conduct a large outward resurgent current at the precise moment to help repolarize the ventricular action potential (Hancox, J. C. et al. (1998) “Time Course And Voltage Dependence Of Expressed HERG Current Compared With Native “Rapid” Delayed Rectifier K Current During The Cardiac Ventricular Action Potential,” Pflugers Arch 436:843-853; Zhou, Z. et al. (1998) “Properties Of HERG Channels Stably Expressed In HEK 293 Cells Studied At Physiological Temperature,” Biophys. J. 74:230-241). The significance of HERG and I_{Kr} kinetics is that they allow for the plateau phase of the AP by being mostly inactivated, but channels are primed to resurge and conduct an outward current for the late repolarization phase of the action potential. The relatively slow deactivation rate of the channels (FIG. 2, underlined arrow) is critical for the resurgent current and repolarization of the cardiac action potential.

The recoveries from inactivation and deactivation transitions are critical determinants of the resurgent HERG current. The mechanism underlying rapid inactivation and recovery from inactivation in HERG depends on residues near the outer mouth of the channel pore (Herzberg, I. M. et al. (1998) “Transfer Of Rapid Inactivation And E-4031 Sensitivity From HERG to M-EAG Channels,” J. Physiol. 511(1):3-14; Schonherr, R. et al. (1996) “Molecular Determinants For Activation And Inactivation of HERG, A human Inward Rectifier Potassium Channel,” J. Physiol. (Lond) 493: 635-642; Smith, P. L. et al. (1996) “The Inward Rectification Mechanism Of The HERG Cardiac Potassium Channel” Nature 379:833-836) and is similar to “C-type” inactivation in Shaker K⁺ channels, in which the outer pore is thought to constrict (Baukrowitz, T. et al. (1995) “Modulation Of K⁺ Current By Frequency And External [K+]: A Tale Of Two inactivation Mechanisms,” Neuron 15:951-960; Hoshi, T. (1991) “Two Types Of Inactivation In Shaker K⁺ Channels: Effects Of Alterations In The Carboxy-Terminal Region,” Neuron 7:547-556; Ogielska, E. M. et al. (1995) “Cooperative Subunit Interactions In C-Type Inactivation Of K Channels,” Biophys J69: 2449-2457).

The significance of fundamental gating mechanisms in HERG is highlighted by mutations associated with LQT2. In particular, a set of LQT2-associated mutations occur in regions critical for slow deactivation gating in HERG channels. For example, many mutations are found in the PAS domain (http://www.fsm.it/cardmoc/). HERG channels bearing LQT2 mutations in the HERG PAS domain found that mutant channels had faster deactivation kinetics than in wild-type channels, implying that the mechanism of slow deactivation was disrupted by mutations in the PAS domain (Chen, J. et al. (1999) “Long QT Syndrome-Associated Mutations In The Per-Arnt-Sim (PAS) Domain of HERG Potassium Channels Accelerate Channel Deactivation,” J. Biol. Chem. 274:10113-10118). The association of LQTS phenotypes with specific HERG mutations that disrupt slow deactivation implies a link between slow deactivation gating, I_{Kr} function in vivo and lie-threatening cardiac arrhythmias. In computational models of action potential formation, using HERG kinetics that mimicked those of a channel with a PAS domain mutation (HERG R56Q) and fast deactivation, the action potential was prolonged an in the model due to the faster kinetics of HERG R56Q (Clancy, C. E. et al. (2001) “Cellular Consequences Of HERG Mutations In The Long QT Syndrome: Precursors To Sudden Cardiac Death,”Cardiovasc. Res. 50:301-313). Thus, there is a crucial physiological and pathophysiological role for HERG gating, and HERG slow deactivation gating in particular, in heart.

Deactivation in HERG channels is in part modulated by the N-terminal region of the protein (Morais Cabral, J. H. et al. (1998) “Crystal Structure And Functional Analysis Of The HERG Potassium Channel N Terminus: A Eukaryotic PAS Domain,” Cell 95:649-655; Spector, P. S. et al. (1996) “Fast Inactivation Causes Rectification Of The I_{Kr} Channel,” J. Gen. Physiol. 107:611-619; Wang, J. et al. (1998) “Regulation Of Deactivation By An Amino Terminal Domain In Human Ether-A-Go-Go-Related Gene Potassium Channels,” J. Gen. Physiol. 112:637-647 [published erratum appears in J. Gen. Physiol. 113(2):359 (1999)]. In HERG, like other voltage-activated K⁺ channels, voltage-dependent opening and closing (activation and deactivation gating) is mediated by the charged S4 voltage-sensor region (Hille, B. (2001) “Ion Channels Of Excitable Membranes, 3rd ed. Sinauer Associates, Sunderland; Piper, D. R. et al. (2003) “Gating Currents Associated With Intramembrane Charge Displacement In HERG Potassium Channels,” Proc. Natl. Acad. Sci. (USA) 100:10534-10539; Smith, P. L. et al. (2002) “Fast And Slow Voltage Sensor Movements In HERG Potassium Channels,” J. Gen. Physiol. 119: 275-293; Zhang, M. et al. (2004) “Gating Charges In The Activation And Inactivation Processes Of The HERG Channel,” J. Gen. Physiol. 124:703-718). Movement of the S4 is coupled to the opening of the channel pore. Deletions of amino acids 2-9 or 2-16 at the extreme N-terminal region speed the deactivation rate (Morais Cabral, J. H. et al. (1998) “Crystal Structure And Functional Analysis Of The HERG Potassium Channel N Terminus: A Eukaryotic PAS Domain,” Cell 95:649-655; Wang, J. et al. (1998) “Regulation Of Deactivation By An Amino Terminal Domain In Human Ether-A-Go-Go-Related Gene Potassium Channels,” J. Gen. Physiol. 112:637-647 [published erratum appears in J. Gen. Physiol. 113(2):359 (1999)]. Re-addition of a peptide composed of amino acids 1-16 to the face of an excised patch expressing HERG Δ2-354 (a HERG channel having a deletion of the entire N-terminal region), partially restored slow deactivation and was quickly reversible (Wang, J. et al. (2000) “Dynamic Control Of Deactivation Gating By A Soluble Amino-Terminal Domain In HERG K(⁺) Channels,” J. Gen. Physiol. 115:749-758). The PAS CAP domain in the HERG-1a N-terminal region also regulates deactivation.

Accordingly, HERG channels bearing engineered deletions of the N-terminal region (HERG N_{Del}) have deactivation kinetics that are approximately 10-fold faster than the deactivation kinetics of wild-type ERG-1a channels (Wang, J. et al. (1998) “Regulation Of Deactivation By An Amino Terminal Domain In Human Ether-A-Go-Go-Related Gene Potassium Channels,” J. Gen. Physiol. 112:637-647 [published erratum appears in J. Gen. Physiol. 113(2):359 (1999)]; Viloria, C. G. (2000) “Differential Effects Of Amino-Terminal Distal And Proximal Domains In The Regulation Of Human Erg K(⁺) Channel Gating Biophys,” J. 79(1):231-246). In contrast, channel activation (representing the closed to open transitions) is not markedly different in HERG N_{Del} when compared to wild-type HERG-1a channels. Thus, in HERG-1a channels, the N-terminal region appears to specifically modulate channel deactivation (i.e., the open to closed transitions) by a novel auto-excitatory mechanism that can be diagrammed as follows (Scheme 1):

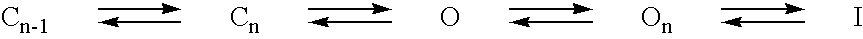

In this scheme, a second open state (O_{N}) is proposed to account for the effect of the N-terminal region on slow deactivation and its apparent lack of effect of the N-terminal region on the C to O transitions that make up channel activation gating. The transition from O_{N} to O is slow as depicted in Scheme 1.

The molecular mechanism of slow deactivation gating in HERG has not previously been completely understood. Some domains of HERG have been identified as playing a role in slow deactivation. The first 135 amino acids of the intracellular N-terminal region of HERG (i.e., the HERG-1a NH₂-Terminal Domain) are necessary for slow deactivation gating (Morais Cabral, J. H. et al. (1998) “Crystal Structure And Functional Analysis Of The HERG Potassium Channel N Terminus: A Eukaryotic PAS Domain,” Cell 95:649-655). These first 1-135 amino acids are conserved among the Eag (ether á go-go) family of channels, which includes HERG (Warmke, J. W. et al. (1994) “A Family Of Potassium Channel Genes Related To Eag In Drosophila And Mammals,” Proc. Natl. Acad. Sci. (USA) 91(8):3438-3442).

Channels lacking the HERG-1a NH₂-Terminal Domain have fast deactivation kinetics (Morais Cabral, J. H. et al. (1998) “Crystal Structure And Functional Analysis Of The HERG Potassium Channel N Terminus: A Eukaryotic PAS Domain,” Cell 95:649-655; Warmke, J. W. et al. (1994) “A Family Of Potassium Channel Genes Related To Eag In Drosophila And Mammals,” Proc. Natl. Acad. Sci. (USA) 91(8):3438-3442). Re-application of a peptide encoding the HERG-1a NH₂-Terminal Domain to channels with an engineered deletion of the domain partially restored slow deactivation gating (Morais Cabral, J. H. et al. (1998) “Crystal Structure And Functional Analysis Of The HERG Potassium Channel N Terminus: A Eukaryotic PAS Domain,” Cell 95:649-655). This finding suggested that the HERG-1a NH₂-Terminal Domain interacts with the other regions of the channel to produce slow deactivation gating. The 3-dimensional structure of the HERG-1a NH₂-Terminal Domain was solved by X-ray crystallography. The structure showed that amino acids 26-135 had a structure similar to the structure of the PAS family of effector proteins. Deletions in the region upstream of the PAS domain also disrupted deactivation (Morais Cabral, J. H. et al. (1998) “Crystal Structure And Functional Analysis Of The HERG Potassium Channel N Terminus: A Eukaryotic PAS Domain,” Cell 95:649-655; Wang, J. et al. (1993) “Comparative Mechanisms Of Antiarrhythmic Drug Action In Experimental Atrial Fibrillation. Importance Of Use-Dependent Effects On Refractoriness,” Circulation 88: 1030-1044). A peptide corresponding to the short upstream region encoding amino acids 1-16 partially restored slow deactivation to HERG channels lacking the N-terminal region (Wang, J. et al. (2000) “Dynamic Control Of Deactivation Gating By A Soluble Amino-Terminal Domain In HERG K(⁺) Channels,” J. Gen. Physiol. 115:749-758). Thus, the short region upstream of PAS binds directly to HERG channels. Short regions flanking PAS domains are found in other PAS-containing proteins (Teng G. Z. X. et al. (2004) “Prolonged Repolarization And Triggered Activity Induced By Adenoviral Expression Of HERG N629D In Cardiomyocytes Derived From Stem Cells,” Cardiovasc. Res. 61 :268-277). Since these regions ‘cap’ the PAS domains they are termed “PAS-CAP” regions. HERG-PAS CAP is thought to transiently bind to the HERG channel, since the slowing effect of the HERG-PAS CAP peptide depends on the presence of the peptide. In contrast, with re-introduction of the HERG-1a N-Terminal Domain, the partial restoration of deactivation persists, suggesting a more stable interaction with the channel. Thus, the PAS-CAP domain makes a transient interaction with the channel and the PAS domain makes a stable interaction with the channel. In wild-type HERG-1a channels, both the PAS-CAP and PAS domain interactions with the channel are necessary for slow deactivation gating. The PAS domain thus plays a role in keeping the PAS-CAP region at a high local concentration so that the PAS-CAP region can interact with the core of the channel.

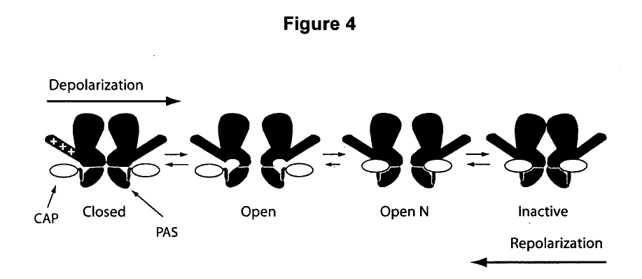

The interactions of the PAS and PAS-CAP domains with the intracellular regions of the channel are summarized in FIG. 4. In Closed channels the voltage sensor (S3-S4 paddle region, +symbols) is in a resting state. In the Closed state the PAS domain is bound to the channel directly, but the CAP region is not. With depolarization, channels are driven into an Open state due to movement of the voltage-sensor. In the Open state a binding site for PAS-CAP region is uncovered and the PAS-CAP region interacts with the channel (Open N). With repolarization, return from the Open N state is slow (arrow) due to a favorable PAS-CAP interaction with the channel. With depolarization from the Open N state channels enter an Inactive state. The Inactive state is not critical for slow deactivation; channels with deletions that abolish inactivation gating retain wild-type-like slow deactivation. The schematic is simplified, since when the Open N state is disrupted channels still can enter the Inactive state. The precise pathway to the Inactive state does not affect the general conclusion that PAS-CAP binding slows the deactivation rate of the channel.

Rat Eag channels contain a PAS-CAP region that appears to interact with the S4 region to regulate the voltage-dependent movement between channel closed states (Cole-Moore shift) that is characteristic of Eag channels (Terlau, H. et al. (1997) “Amino Terminal-Dependent Gating Of The Potassium Channel Rat eag Is Compensated By A Mutation In The S4 Segment,” J. Physiol. (Lond) 502:537-543). The evidence for this interaction is that a small deletion in the rEAG PAS-CAP region was compensated by mutating a His residue to an Arg residue at the base of the S4:

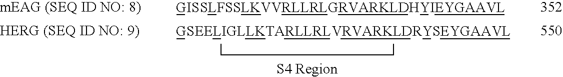

However, HERG channels do not exhibit a Cole-Moore shift as seen in rEag (Trudeau, M. C. et al. (1999) “Functional Analysis Of A Mouse Brain Elk-type K⁺ Channel,” J. Neurosci. 19:2906-2918) and rEag channels do not exhibit slow deactivation as seen in HERG (Robertson, G. A. et al. (1996) “Potassium currents Expressed From Drosophila And Mouse eag cDNAs in Xenopus Oocytes,” Neuropharmacology 35:841-850; Terlau, H. et al. (1997) “Amino Terminal-Dependent Gating Of The Potassium Channel Rat eag Is Compensated By A Mutation In The S4 Segment,” J. Physiol. (Lond) 502:537-543.

One aspect of the present invention derives from the recognition that HERG employs a comparable interaction between the PAS-CAP and the base of S4 as in rEAG, but that in this interaction imparts slow deactivation in HERG-1a channels. A second aspect of the present invention derives from the recognition that PAS-CAP binding determines slow deactivation and that the physical basis for such slow deactivation gating is an electrostatic interaction between members of a cluster of positively charged residues in PAS-CAP with negatively charged residues in the channel that alters the return of the voltage-sensor. Thus, charged residues in the PAS-CAP region play a necessary role in HERG channel repolarization, and provide a target for therapeutic intervention.

Such recognitions permit the identification of the sites in the core regions of HERG that affect slow deactivation. Although Weerapura, M. et al. (2002) (“A Comparison Of Currents Carried By HERG, With And Without Coexpression Of MiRP1, And The Native Rapid Delayed Rectifier Current. Is MiRP1 The Missing Link?” J. Physiol. (Lond) 540:15-27) used scanning cysteine-mutagenesis in the S4-S5 linker region and a G546C alteration to show that the S4-S5 linker region (FIG. 1) was involved in slow deactivation gating, a direct interaction between the S4-S5 linker region and the PAS domain was not established. The present invention uses the structure of the HERG-PAS domain to obtain insight into the characteristics of the PAS receptor. The PAS domain appears to interact with the core of the HERG channel via hydrophobic residues on the surface of PAS (see, O'Leary, M. E. (2001) “Inhibition Of Human Ether-A-Go-Go Potassium Channels By Cocaine,” Mol. Pharmacol. 59:269-77). Analysis of the 3-dimensional structure of HERG-PAS reveals the presence of a group of hydrophobic residues on the surface. Mutation of either of two hydrophobic residues (Y43 and F29) disrupted slow deactivation, whereas, notably, mutations at other surface sites did not disrupt deactivation (see, O'Leary, M. E. (2001) “Inhibition Of Human Ether-A-Go-Go Potassium Channels By Cocaine,” Mol. Pharmacol. 59:269-77). The present invention establishes the importance of the Y43 and F29 hydrophobic residues for PAS domain function.

One aspect of the present invention derives from the present invention's finding that the hydrophobic surface of the PAS domain interacts with hydrophobic residues in a PAS-receptor site located in the core of the channel that includes residues in the S4-S5 linker, S5 and S6 regions, and relates to new methods and reagents for identifying the determinants of the PAS receptor site. More specifically, the invention relates to the recognition that a fragment of HERG is able to bind to the channel and can therefore be employed as a probe of the receptor site.

### C. Effectors of HERG-1a Function

The deactivation kinetics of cardiac I_{Kr} channels measured in native cells are significantly faster than the deactivation kinetics of HERG-1a channels measured in heterologous systems (Sanguinetti, M. C. et al. (1995) “A Mechanistic Link Between An Inherited And An Acquired Cardiac Arrhythmia: HERG Encodes The I_{Kr} Potassium Channel,” Cell 81(2):299-307; Sanguinetti, M. C. et al. (1990) “Two Components Of Cardiac Delayed Rectifier K⁺ Current. Differential Sensitivity To Block By Class III Antiarrhythmic Agents,” J. Gen. Physiol. 96:195-215; Weerapura, M. et al. (2002) “A Comparison Of Currents Carried By HERG, With And Without Coexpression Of MiRP1, And The Native Rapid Delayed Rectifier Current. Is MiRP1 The Missing Link?” J. Physiol. (Lond) 540: 15-27). In a side-by-side study of HERG-1a and native I_{Kr} from guinea pig myocardium, the kinetics deactivation at −50 mV of HERG-1a homomers and native I_{Kr} was 788 ms versus 319 ms, that is, the kinetics HERG-1a were significantly slower than those of native I_{Kr} (Weerapura, M. et al. (2002) “A Comparison Of Currents Carried By HERG, With And Without Coexpression Of MiRP1, And The Native Rapid Delayed Rectifier Current. Is MiRP1 The Missing Link?” J. Physiol. (Lond) 540:15-27). The identical recording conditions used to study HERG-1a and native I_{Kr} suggested that there is a bonafide difference in the deactivation kinetics of native I_{Kr} and HERG-1a channels. The results imply that other proteins also comprise I_{Kr} or that I_{Kr} deactivation is differently modulated than HERG-1a.

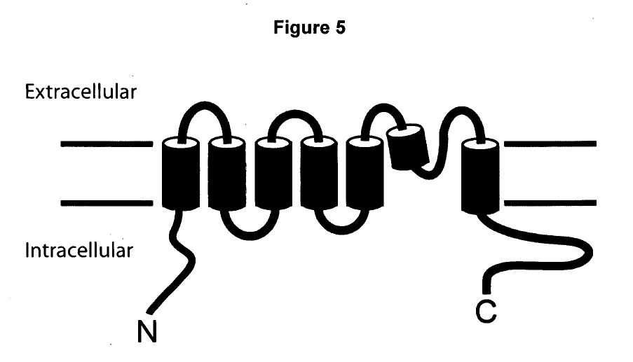

One aspect of the present invention is that HERG-1b is responsible for the faster deactivation measured in vivo. Unlike the long N-terminal region of HERG-1a channels, HERG-1b channels have a short N-terminal region of 56 amino acids. The initial 36 amino acids of the ERG1b N-terminal region are novel, but the remaining N-terminal region and the rest of ERG1b is identical to ERG1a. Consequently, ERG-1b lacks the PAS domain and PAS-CAP region (FIG. 5). Due to the lack of these N-terminal regions, HERG-1b channels expressed in Xenopus oocytes had deactivation kinetics that were approximately 10-fold faster than HERG-1a or mouse ERG-1a (MERG-1a) kinetics (London, B. et al. (1997) “Two Isoforms Of The Mouse Ether-A-Go-Go-Related Gene Coassemble To Form Channels With Properties Similar To The Rapidly Activating Component Of The Cardiac Delayed Rectifier K⁺ Current,” Circ. Res. 81(5):870-878).

Co-expression of a mixture of ERG-1a and ERG-1b subunits in a heterologous system revealed that subunits formed heteromeric channels and that the deactivation kinetics of mixtures of ERG-1a/ERG-1b channels mimic the kinetics of native channels (London, B. et al. (1997) “Two Isoforms Of The Mouse Ether-A-Go-Go-Related Gene Co-Assemble To Form Channels With Properties Similar To The Rapidly Activating Component Of The Cardiac Delayed Rectifier K⁺ Current,” Circ. Res. 81(5):870-878). In native myocytes, HERG-1a and HERG-1b can form biochemical interactions. The present invention recognizes that this finding indicates that at least some of the I_{Kr} channels in the human heart are heteromeric channels composed of subunits of both HERG-1a and HERG-1b. ERG-1b is detected with immunocytochemistry at the T-tubules, as is HERG-1a (Jones, E. M. et al. (2004) “Cardiac I_{Kr} Channels Minimally Comprise hERG1a and 1b subunits,” J. Biol. Chem. 279:44690-44694). Mice bearing a knock-out of the MERG1b gene had slower kinetics of I_{Kr} in fetal mice, but interestingly, I_{Kr} was completely absent in cells from the adult knock-out mouse (Lees-Miller, J. P. et al. (2003) “Selective Knockout of Mouse ERG1 B Potassium Channel Eliminates I_{(Kr)} In Adult Ventricular Myocytes And Elicits Episodes Of Abrupt Sinus Bradycardia,” Mol. Cell. Biol. 23:1856-18562). However, in mouse heart, I_{Kr} is unlikely to repolarize the terminal phase of the action potential (Lees-Miller, J. P. et al. (2003) “Selective Knockout of Mouse ERG1 B Potassium Channel Eliminates I_{(Kr)} In Adult Ventricular Myocytes And Elicits Episodes Of Abrupt Sinus Bradycardia,” Mol. Cell. Biol. 23:1856-18562) and so using mouse heart as a model system to study the relationship between I_{Kr} slow deactivation and repolarization of the AP has serious shortcomings.

Exploitation of the possible functional relationship between HERG-1a and HERG-1b has been precluded by the inability to determine whether the faster deactivation kinetics measured for I_{Kr} are in fact caused by HERG-1b. One aspect of the present invention is the development of a specific probe of HERG-1a, and its use to establish the functional relationship between HERG-1a and HERG-1b.

### D. Uses of the Present Invention

The present invention provides ERG proteins, polypeptides, peptides and polynucleotides that have applications in drug discovery and in the diagnosis of diseases and conditions in humans, and in non-human mammals (e.g., dogs, cats, horses, cattle, etc.).

Most preferably, suitable polypeptides and peptides of the invention will comprise a portion of the intracellular amino-terminal region of HERG-1a that is capable of restoring the slow deactivation gating of HERG channels. Preferably, the peptides will comprise at least 10, at least 16, at least 20, at least 40, at least 60, at least 80, or more preferably at least 100 amino acid residues in length and will contain the region of Herg-1a residues 1-135 responsible for restoring slow deactivation gating of HERG channels. Most preferably, the peptide will comprise at least 10, at least 20, at least 30, at least 40, at least 50, at least 60, at least 70, at least 80, at least 90, or more preferably at least 99 contiguous amino acid residues of the HERG-1a Amino Terminal Domain. Preferably the polypeptides will comprise at least 100, at least 110, or still more preferably at least 120 amino acid residues in length and will contain the region of Herg-1a residues 1-135 responsible for restoring slow deactivation gating of HERG channels. Most preferably, the polypeptide will comprise the “HERG-1a Amino Terminal Domain,” or a peptide portion of such Domain having at least 10, at least 20, at least 30, at least 40, at least 50, at least 60, at least 70, at least 80, at least 90, or more preferably at least 100 contiguous amino acid residues of the HERG-1a Amino Terminal Domain. Most preferably, suitable polynucleotides of the present invention will comprise a portion of the nucleotide sequences disclosed herein that encode the desired ERG proteins, polypeptides, peptides. Alternatively, any polynucleotide that encodes such desired ERG proteins, polypeptides, peptides may be employed.

1. Applications to Methods of Drug Discovery

(A) Antiarrhythmic Drugs

The blockade of HERG currents causes lengthening of the cardiac action potential, which may produce a beneficial class III antiarrhythmic effect (Thomas, D. et al. (2006) “The Cardiac hERG/IKr Potassium Channel as Pharmacological Target: Structure, Function, Regulation, and Clinical Applications,” Curr. Pharmaceut. Des. 12:2271-2283; Hondeghem, L. M. et al. (1990) “Class III Antiarrhythmic Agents Have A Lot Of Potential But A Long Way To Go—Reduced Effectiveness And Dangers Of Reverse Use-Dependence,” Circulation 81:686-90. Antagonists of HERG-1b are thus of particular importance as potential anti-arrhythmic drugs.

The finding of the present invention that the HERG-1a Amino Terminal Domain can be employed as a specific functional probe of HERG-1b to determine whether a potential anti-arrhythmic drug acts as an antagonist of HERG-1b function. For example, the I_{Kr} current of a membrane having HERG channels (either an in vitro membrane or the membrane of a cell) is determined in the presence of the HERG-1a Amino Terminal Domain and a candidate agent, and the effect on the I_{Kr} current is compared to that observed in the absence of the HERG-1a Amino Terminal Domain. The detection of a differential effect indicates that the candidate agent is a specific antagonist of HERG-1b.

HERG-1a isoform 3 is abundantly present in heart cells (Kupershmidt, S. et al. (1998) “A K⁺ Channel Splice Variant Common In Human Heart Lacks A C-Terminal Domain Required For Expression Of Rapidly Activating Delayed Rectifier Current,” J. Biol. Chem. 273(42):27231-27235). The HERG-1a Carboxy Terminal Domain can be employed as a specific functional probe of HERG-1a isoform 3 or isoform 4 to determine whether a potential anti-arrhythmic drug acts as an antagonist of HERG-1a isoform 3 function or HERG-1a isoform 4 function. For example, the I_{Kr} current of a membrane having HERG channels (either an in vitro membrane or the membrane of a cell) is determined in the presence of the HERG-1a Carboxy Terminal Domain and a candidate agent, and the effect on the I_{Kr} current is compared to that observed in the absence of the HERG-1a Carboxy Terminal Domain. The detection of a differential effect indicates that the candidate agent is a specific antagonist of HERG-1a isoform 3.

Although rat myocytes may be employed for HERG and I_{Kr} experiments in native myocytes, for I_{Kr} determinations, a preferred cell type is the guinea pig myocyte. Guinea pig myocytes have a measurable I_{Kr} current and were the cell type in which I_{Kr} was first identified. The discovery of the specific I_{Ks} channels blocker makes it possible to directly measure I_{Kr} (Weerapura, M. et al. (2002) “A Comparison Of Currents Carried By HERG, With And Without Coexpression Of MiRP1, And The Native Rapid Delayed Rectifier Current. Is MiRP1 The Missing Link?” J. Physiol. (Lond) 540: 15-27). It is preferred to employ a system in which I_{Kr} repolarizes the ventricular action potential, as is the role of I_{Kr} in guinea pig ventricular myocytes (Sanguinetti, M. C. et al. (1990) “Two Components Of Cardiac Delayed Rectifier K⁺ Current. Differential Sensitivity To Block By Class III Antiarrhythmic Agents,” J. Gen. Physiol. 96:195-215). Of further importance is that the action potential in guinea pig myocytes is characteristic in shape and duration and is prolonged when I_{Kr} is blocked (Sanguinetti, M. C. et al. (1990) “Two Components Of Cardiac Delayed Rectifier K⁺ Current. Differential Sensitivity To Block By Class III Antiarrhythmic Agents,” J. Gen. Physiol. 96:195-215).

(B) Non-Antiarrhythmic Drugs

HERG channels are inhibited by a variety of non-antiarrhythmic compounds (Redfern, W. S. et al. (2003) “Relationships Between Preclinical Cardiac Electrophysiology, Clinical QT Interval Prolongation And Torsade De Pointes For A Broad Range Of Drugs: Evidence For A Provisional Safety Margin In Drug Development,” Cardiovasc. Res. 58:32-45), such as the tricyclic antidepressants imipramine and amitriptyline, the selective serotonin reuptake inhibitor fluoxetine, the histamine receptor antagonists terfenadine and astemizole, the antiestrogen tamoxifen, fluoroquinolone antibacterial drugs, the anticancer agent amsacrine and the antipsychotic drugs haloperidol and chlorpromazine (Thomas, D. et al. (2006) “The Cardiac hERG/IKr Potassium Channel as Pharmacological Target: Structure, Function, Regulation, and Clinical Applications,” Curr. Pharmaceut. Des. 12:2271-2283).

Sudden death as a side effect of the action of non-antiarrhythmic drugs is a major pharmacological safety concern facing the pharmaceutical industry (Aronov, A. M. (2005) “Predictive in silico Modeling For hERG Channel Blockers,” Drug Discov. Today 10(2):149-55; Jamieson, C. et al. (2006) “Medicinal Chemistry of hERG Optimizations: Highlights and Hang-Ups,” J. Medicin. Chem. 49(17):5029-5046 ). Multiple drugs (e.g., Astemizole, Sertindole, Terfenadine, Grepafloxacin and Cisapride) have been withdrawn from the market due to reports of sudden cardiac death. Significantly, safety issues with such drugs have been linked to an undesired blockade of cardiac I_{Kr} current associated with the abnormal HERG structure or expression observed in patients having LQTS or its non-hereditary variant. As a consequence, early detection of compounds that mediate an undesired blockade of HERG K⁺ channels has become an important objective of the pharmaceutical industry (Aronov, A. M. (2005) “Predictive in silico Modeling For hERG Channel Blockers,” Drug Discov. Today 10(2):149-55).

The present invention, by identifying the sites required for ERG-1 channel function provides assays that may be used to identify candidate pharmacological agents that induce undesirable effects on the cardiac I_{Kr} current. For example, computer modeling can be used to assess whether a candidate agent unacceptably interferes with the interaction of PAS residues to the S4/S5 linker and S6 domains identified as relevant to ERG function. Alternatively, such agents may be introduced to a membrane having HERG-1b channels (either an in vitro membrane or the membrane of a cell) in the presence of the HERG-1a N-Terminal Domain, and the effect of the agent's presence on the restoration of slow deactivation current ascertained. Agents that affect the ability of the HERG-1a N-Terminal Domain to restore slow deactivation current have a HERG blocking activity. Such activity can be compared with that of known HERG blocking agents (e.g., Astemizole, Sertindole, Terfenadine, Grepafloxacin and Cisapride) to determine whether such activity is of sufficient magnitude to preclude further development of the candidate agent. Such assays can comprise fluorescence-based assays (Netzer, R. et al. (2001) “Screening Lead Compounds For QT Interval Prolongation,” Drug Discov. Today 6:78-84; Friesen, R. W. et al. (2003) “Optimization Of A Tertiary Alcohol Series Of Phosphodiesterase-4 (PDE4) Inhibitors: Structure-Activity Relationship Related To PDE4 Inhibition And Human Ether-Ago-Go Related Gene Potassium Channel Binding Affinity,” J. Med. Chem. 46:2413-2426; McCauley, J. A. et al. (2004) “NR2B-Selective N-Methyl-D-Aspartate Antagonists: Synthesis And Evaluation Of5-Substituted Benzimidazoles,” J. Med. Chem. 47: 2089-2096). Alternatively, automated high throughput patch clamp assays (e.g. planar patch technology) may be employed (Wood, C. et al. (2004) “Patch Clamping by Numbers,” Drug Discov. Today 9:434-441).

2. Diagnostic Applications

(A) Diagnosis of Abnormal ERG Channel Dysfunction or Composition

The response of individual patients to pharmacotherapy has been found to be associated with the presence of mutations or polymorphisms in HERG 1 or HERG-1b subunits (Thomas, D. et al. (2006) “The Cardiac hERG/IKr Potassium Channel as Pharmacological Target: Structure, Function, Regulation, and Clinical Applications,” Curr. Pharmaceut. Des. 12:2271-2283).

The diagnosis of abnormal ERG channel composition has been hampered by several factors, including the similarity of ERG-1b and ERG-1a, and the lack of specific blockers of ERG-1b that can be used to selectively identify functioning ERG-1b channels in vivo. The finding of the present invention that the HERG-1a Amino Terminal Domain can be employed as a specific functional probe of HERG-1b permits one to determine the ERG channel composition of a human and non-human mammalian patients, and to thereby diagnose diseases and conditions that reflect abnormal ERG channel composition. For example, the I_{Kr} current of a patient's ERG channel is determined in the presence and absence of an ERG-1a Amino Terminal Domain (e.g., for humans, the HERG-1a Amino Terminal Domain), and the effect on the I_{Kr} current is compared to that observed with normal ERG channels. In an analogous manner, the I_{Kr} current of a patient's ERG channels can be determined in the presence and absence of ERG-1b or the ERG-1b Amino Terminal or Carboxy Terminal Domain (e.g., for humans, HERG-1b or the HERG-1b Amino Terminal or Carboxy Terminal Domain), and the effect on the I_{Kr} current compared to that observed with ERG channels of normal cells. Such assays may be performed using a membrane having such ERG channels or using a cell whose cellular membrane has such channels. In one embodiment such ERG channels are obtained by cloning and expressing the ERG-1 subunits of the patient. Alternatively, suitable cells can be isolated by biopsy.

Such assays may be performed in order to assess a patient's suitability for a particular pharmacological agent prior to its initial selection or in concert with its use to assess whether undesired consequences to such therapy have developed.

(B) Diagnosis of Cancer

ERG, and in particular, HERG, has been found to play a role in numerous forms of cancer, including endometrial cancer, colorectal adenocarcinomas, and acute myeloid and lymphoid leukemias (Arcangeli, A. (2005) “Expression And Role Of HERG Channels In Cancer Cells,” Novartis Found. Symp. 266:225-232; Witchel, H. J. (2007) “The hERG Potassium Channel As A Therapeutic Target,” Expert Opin Ther. Targets 11(3):321-336).

The ability of the ERG proteins, polypeptides, peptides and polynucleotides of the present invention to measure the function of individual ERG subunits provides a means for evaluating the involvement, staging, prognosis and amenability to treatment of a patient's cancer (relative to cancers that involve or do not involve ERG. For example, the ERG proteins of a human patient can be used to form channels in the presence and absence of the HERG-1a Amino Terminal or Carboxy Terminal Domains, and the impact of such Domain on I_{Kr} assessed in order to determine the extent of HERG channel dysfunction or the subunit constituent profile of the patient's HERG channels. Such information, in concert with information from normal cells, cancer cells responding to therapy and cancer cells refractile to therapy provide means for diagnosing cancer, and for assessing the staging of cancer cells.

3. Therapeutic Methods

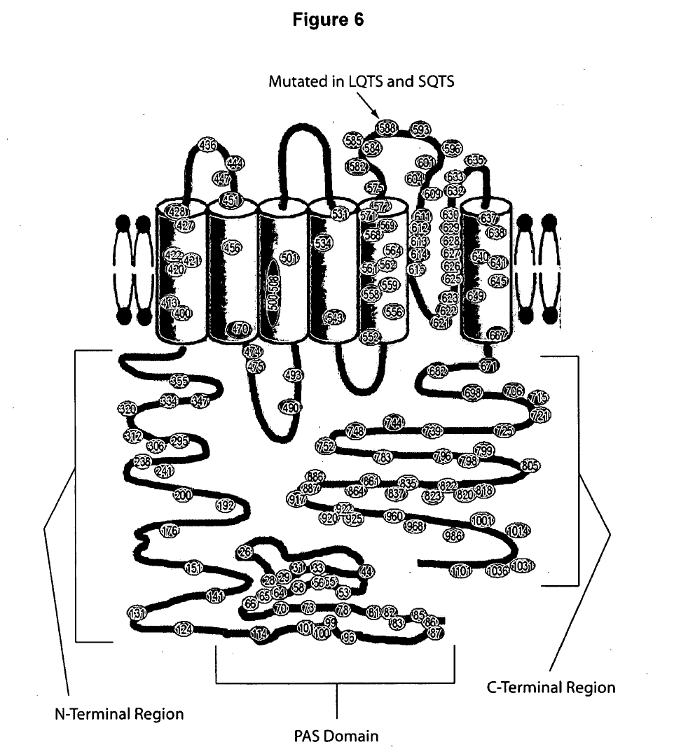

Modulation of K⁺ channel activity offers therapeutic advantage in two primary settings:
(i) as a process of influencing stability of the cell irrespective of the cause of instability and
(ii) as a mechanism to rectify the pathophysiological state related to a channelopathy (Lawson, K. (2006) “Modulation of Potassium Channels as a Therapeutic Approach,” Current Pharmaceut. Des. 12:459-470).
   In particular, the invention relates to the provision of ERG domains that either increase or decrease the deactivation kinetics of I_{Kr}. For example, Long QT Syndrome is characterized by fast deactivation kinetics. Certain mutations associated with Long QT Syndrome are depicted in FIG. 6. In accordance with the principles of the present invention, provision of the Amino Terminal Region of HERG-1a, or of the Carboxy Terminal Region of HERG-1a would decrease (and hence normalize) the deactivation kinetics of cardiac cells of a patient having a hereditary or acquired Long QT Syndrome. Conversely, Short QT Syndrome is characterized by abnormally slow deactivation kinetics. In accordance with the principles of the present invention, provision of HERG-1b, or HERG-1a isoform 3, or HERG-1a isoform 4, or a polypeptide or peptide fragment of such proteins would increase (and hence normalize) the deactivation kinetics of cardiac cells of a patient having a hereditary or acquired Short QT Syndrome.

(A) Genetic Therapy to Alter ERG Channel Composition or Function

In one embodiment, such provision is achieved through genetic therapy by administering to a patient in need of such intervention one or more therapeutic polynucleotides that encode one or more ERG proteins, polypeptides or peptides (as defined above) so as to modulate (increase or decrease) I_{Kr} by altering ERG channel composition or function. Accordingly, and in one aspect, the invention provides methods for preventing or treating cardiac arrhythmia. Preferably, such modulation in I_{Kr} will normalize the deactivation kinetics of a recipient cell by at least 10%, more preferably by at least 20%, and still more preferably by at least 50%. Methods of cardiac genetic therapy that may be employed to accomplish this goal are disclosed by: U.S. Pat. Nos. 7,034,008; 6,992,070; 6,867,196; 6,852,704; Fishbein, I. et al. (2005) “Site Specific Gene Delivery In The Cardiovascular System,” J. Control. Release 109(1-3):37-48; Glenn, C. M. et al. (2003) “Gene Therapy To Develop A Genetically Engineered Cardiac Pacemaker,” J. Cardiovasc. Nurs. 8(5):330-336; Praveen, S. V. et al. (2006) “Gene Therapy In Cardiac Arrhythmias,” Indian Pacing Electrophysiol. J. 6(2):111-118; Abraham, M. R. et al. (2005) “Antiarrhythmic Engineering Of Skeletal Myoblasts For Cardiac Transplantation,” Circ. Res. 97(2):159-167; Anantharam, A. et al. (2003) “Pharmacogenetic Considerations In Diseases Of Cardiac Ion Channels,” J. Pharmacol. Exp. Ther. 307(3):831-838; Donahue, J. K. (2007) “Gene Therapy For Cardiac Arrhythmias: A Dream Soon To Come True?” J. Cardiovasc. Electrophysiol. 18(5):553-559; Pachori, A. S. et al. (2006) “Gene Therapy: Role In Myocardial Protection,” Handb. Exp. Pharmacol. 176(Pt 2):335-350; Dib, N. et al. (2006) “The Future Of Cell Therapy For Myocardial Regeneration,” Am. Heart Hosp. J. 4(3):211-215; Bjerregaard, P. et al. (2006) “Targeted Therapy For Short QT Syndrome,” Expert Opin. Ther. Targets 10(3):393-400; Dulak, J. et al. (2006) “New Strategies For Cardiovascular Gene Therapy: Regulatable Pre-Emptive Expression Of Pro-Angiogenic And Antioxidant Genes,” Cell. Biochem. Biophys. 44(1):31-42; Jordan, P. N. et al. (2005) “Therapies For Ventricular Cardiac Arrhythmias,” Crit. Rev. Biomed. Eng. 33(6):557-604; Donahue, J. K. et al. (2005) “Gene Therapy For Cardiac Arrhythmias,” Trends Cardiovasc. Med. 15(6):219-24; all of which methods are herein incorporated by reference.

In preferred embodiments, such administration involves administering at least one of the foregoing polynucleotides with a suitable a myocardium nucleic acid delivery system. In one embodiment, that system includes a non-viral vector operably linked to the polynucleotide. Examples of such non-viral vectors include the polynucleoside alone or in combination with a suitable protein, polysaccharide or lipid formulation. Additionally suitable myocardium nucleic acid delivery systems include viral vector, typically sequence from at least one of an adenovirus (including replication deficient adenovirus), adenovirus-associated virus (AAV), helper-dependent adenovirus, retrovirus, or hemagglutinating virus of Japan-liposome (HVJ) complex. Preferably, the viral vector comprises a strong eukaryotic promoter operably linked to the polynucleotide e.g., a cytomeglovirus (CMV) promoter.

Additionally preferred vectors include viral vectors, fusion proteins and chemical conjugates. Retroviral vectors include moloney murine leukemia viruses and HIV-based viruses. One preferred HIV-based viral vector comprises at least two vectors wherein the gag and pol genes are from an HIV genome and the env gene is from another virus. DNA viral vectors are preferred. These vectors include pox vectors such as orthopox or avipox vectors, herpesvirus vectors such as a herpes simplex I virus (HSV) vector, Adenovirus vectors, and Adeno-associated virus vectors (see, U.S. Pat. No. 7,034,008).

The particular vector chosen will depend upon the target cell and the condition being treated. To simplify the manipulation and handling of the polynucleotides described herein, the nucleic acid is preferably inserted into a cassette where it is operably linked to a promoter. The promoter must be capable of driving expression of the protein in cells of the desired target tissue. Any of a variety of suitable promoters can be employed, such as the cytomegalovirus (CMV) promoter, the Rous sarcoma virus (RSV) promoter, and the MMT promoter. If desired, the polynucleotides of the invention may also be used with a microdelivery vehicle such as cationic liposomes and adenoviral vectors.

The effective dose of the nucleic acid will be a function of the particular expressed protein, the particular cardiac arrhythmia to be targeted, the patient and his or her clinical condition, weight, age, sex, etc.

If desired, the administration step can further include increasing microvascular permeability using routine procedures, typically administering at least one vascular permeability agent prior to or during administration of the gene transfer vector. Examples of particular vascular permeability agents include administration of one or more of the following agents preferably in combination with a solution having less than about 500 micromolar calcium: substance P, histamine, acetylcholine, an adenosine nucleotide, arachidonic acid, bradykinin, endothelin, endotoxin, interleukin-2, nitroglycerin, nitric oxide, nitroprusside, a leukotriene, an oxygen radical, phospholipase, platelet activating factor, protamine, serotonin, tumor necrosis factor, vascular endothelial growth factor, a venom, a vasoactive amine, or a nitric oxide synthase inhibitor.

(B) Protein and Protein Mimetics

In an alternative embodiment, such modulation is achieved by administering to a patient in need of such intervention one or more therapeutic ERG proteins, polypeptides and peptides (as defined above) so as to modulate (increase or decrease) I_{Kr} by altering ERG channel composition or function. Preferably, such modulation in I_{Kr} will normalize the deactivation kinetics of a recipient cell by at least 10%, more preferably by at least 20%, and still more preferably by at least 50%. In a preferred embodiment, such proteins are delivered directly to cardiac tissue, preferably in liposomes or gelatin hydrogels (see, Shao, Z.-Q. et al. (2006) “Effects of Intramyocardial Administration of Slow-Release Basic Fibroblast Growth Factor on Angiogenesis and Ventricular Remodeling in a Rat Infarct Model,” Circ. J. 70:471-477).

The elucidation of reactive sites in HERG-1a alternatively permits the rational design and use of ERG protein mimetics. As used herein, an ERG protein mimetic is a compound whose chemical groups mimic the three dimensional structure of an ERG binding site. Methods of forming such mimetics are preferably adapted from Davis, J. M. et al. (2007) “Synthetic Non-Peptide Mimetics Of Alpha-Helices,” Chem. Soc. Rev. 36(2):326-334; Eichler, J. (2004) “Rational And Random Strategies For The Mimicry Of Discontinuous Protein Binding Sites,” Protein Pept. Lett. 11(4):281-290; Eguchi, M. et al. (2002) “Design, Synthesis, And Application Of Peptide Secondary Structure Mimetics,” Mini Rev. Med. Chem. 2(5):447-462; Kim, H. O. et al. (2000) “A Merger Of Rational Drug Design And Combinatorial Chemistry: Development And Application Of Peptide Secondary Structure Mimetics,” Comb. Chem. High Throughput Screen. 3(3):167-183; Moore, G. J. (1994) “Designing Peptide Mimetics,” Trends Pharmacol. Sci. 15(4):124-129.

In one embodiment, such compounds are provided in concert with an antiarrhythmic drug or with an antihistamine such as fexofenadine (see, U.S. Pat. No. 7,012,082).

Such compounds are preferably formulated into pharmaceutical formulations for administration. Any of a number of suitable pharmaceutical formulations (e.g., see Remington's Pharmaceutical Sciences, 19^{th} Edition, A. R. Gennaro, ed., Mack Publishing Co., Easton, Pa. (1995), incorporated herein by reference in its entirety) may be utilized as a vehicle for the administration of the compounds of the present invention. Such compounds are preferably administered in “pharmacologically acceptable” amounts in the treatment of HERG-associated diseases or conditions. A composition is said to be “pharmacologically acceptable” if its administration can be tolerated by a recipient patient. The administration of such compounds may be for either a “prophylactic” or “therapeutic” purpose. The compositions of the present invention are said to be administered for a “therapeutic” purpose if the amount administered is physiologically significant to provide a therapy for an actual manifestation of the disease. When provided therapeutically, the compound is preferably provided at (or shortly after) the identification of a symptom of actual disease. The therapeutic administration of the compound serves to attenuate the severity of such disease or to reverse its progress. The compositions of the present invention are said to be administered for a “prophylactic” purpose if the amount administered is physiologically significant to provide a therapy for a potential disease or condition. When provided prophylactically, the compound is preferably provided in advance of any symptom thereof. The prophylactic administration of the compound serves to prevent or attenuate any subsequent advance of the disease.

Such compositions can be administered in conventional solid or liquid pharmaceutical administration forms, for example, as uncoated or (film-) coated tablets, capsules, powders, granules, suppositories or solutions. The active substances can, for this purpose, be processed with conventional pharmaceutical aids such as tablet binders, fillers, preservatives, tablet disintegrants, flow regulators, plasticizers, wetting agents, dispersants, emulsifiers, solvents, sustained release compositions, antioxidants and/or propellant gases. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

The therapeutic compositions obtained in this way typically contain from about 0.1% to about 90% by weight of the active substance. As a general proposition, a dosage from about 0.1 to about 50 mg/kg will have therapeutic efficacy, with still higher dosages potentially being employed for oral and/or aerosol administration. Toxicity concerns at the higher level may restrict intravenous dosages to a lower level such as up to about 10 mg/kg, all weights being calculated based upon the weight of the active base, including the cases where a salt is employed. Typically a dosage from about 0.5 mg/kg to about 5 mg/kg will be employed for intravenous or intramuscular administration. A dosage from about 10 mg/kg to about 50 mg/kg may be employed for oral administration.

4. Delivery of Conjugated Molecules

In certain embodiments, the present invention relates to the delivery of an amino acid sequence of the invention conjugated to, fused with, or otherwise combined with, a peptide known as protein transduction domain (“PTP”). A PTD is a short peptide that facilitates the movement of an amino acid sequence across an intact cellular membrane wherein said amino acid sequence would not penetrate the intact cellular membrane without being conjugated to, fused with, or otherwise combined with a PTD. The conjugation with, fusion to, or otherwise combination of a PTD with a heterologous molecule (including, for example, an amino acid sequence, nucleic acid sequence, or small molecule) is sufficient to cause transduction into a variety of different cells in a concentration-dependent manner. Moreover, when drawn to the delivery of amino acids, it appears to circumvent many problems associated with polypeptide, polynucleotide and drug-based delivery. Without being bound by theory, PTDs are typically cationic in nature causing PTDs to track into lipid raft endosomes and release their cargo into the cytoplasm by disruption of the endosomal vesicle. PTDs have been used for delivery of biologically active molecules, including amino acid sequences (see, for example, Viehl C. T. et al. (2005) “A Tat Fusion Protein-Based Tumor Vaccine For Breast Cancer,” Ann. Surg. Oncol. 12:517-525; Noguchi, H., et al. (2004), “A New Cell-Permeable Peptide Allows Successful Allogeneic Islet Transplantation In Mice,” Nat. Med. 10:305-309 (2004); Fu A. L., et al. (2004) “Alternative Therapy Of Alzheimer's Disease Via Supplementation With Choline Acetyltransferase,” Neurosci. Lett. 368:258-262; Del Gazio Moore et al. (2004) “Transactivator Of Transcription Fusion Protein Transduction Causes Membrane Inversion,” J. Biol. Chem. 279(31):32541-32544; US Application Publication No. 20070105775). For example, it has been shown that TAT-mediated protein transduction can be achieved with large proteins such as beta-galactosidase, horseradish peroxidase, RNAase, and mitochondrial malate dehydrogenase, whereby transduction into cells is achieved by chemically cross-linking the protein of interest to an amino acid sequence of HIV-1 TAT (see, for example, Fawell, S. et al. (1994) “Tat-Mediated Delivery Of Heterologous Proteins Into Cells,” Proc. Natl. Acad. Sci. (U.S.A.) 91(2):664-668 (1994); Del Gazio, V. et al. (2003) “Targeting Proteins To Mitochondria Using TAT,” Mol. Genet. Metab. 80(1-2):170-180 (2003)).

Protein transduction methods encompassed by the invention include an amino acid sequence of the invention conjugated to, fused with, or otherwise combined with, a PTD. In particular embodiments a PTD of the invention includes, for example, the PTD from human transcription factor HPH-1, mouse transcription factor Mph-1, Sim-2, HIV-I viral protein TAT, Antennapedia protein (Antp) of Drosophila, HSV-1 structural protein Vp22, regulator of G protein signaling R7, MTS, polyarginine, polylysine, short amphipathic peptide carriers Pep-1 or Pep-2, and other PTDs known to one of ordinary skill in the art or readily identifiable to one of ordinary skill in the art (see, for example, US Application Publication No. 20070105775). One of ordinary skill in the art could routinely identify a PTD by, for example, employing known methods in molecular biology to create a fusion protein comprising a potential PTD and, for example, green fluorescent protein (PTD-GFP) and detecting whether or not GFP was able to transduce a cellular membrane of intact cells, which can be determined by, for example, microscopy and the detection of internal fluorescence. It is noted that the particular PTD is not limited by any of the foregoing and the invention encompasses any known, routinely identifiable, and after-arising PTD.

Methods of protein transduction are known in the art and are encompassed by the present invention (see, for example, Noguchi, H. et al. (2006) “Protein Transduction Technology: A Novel Therapeutic Perspective,” Acta Med. Okayama 60: 1-11; Wadia, J. S. et al. (2002) “Protein Transduction Technology,” Curr. Opin. Biotechnol. 13:52-56; Viehl C. T. et al. (2005) “A Tat Fusion Protein-Based Tumor Vaccine For Breast Cancer,” Ann. Surg. Oncol. 12:517-525; Noguchi, H., et al. (2004), “A New Cell-Permeable Peptide Allows Successful Allogeneic Islet Transplantation In Mice,” Nat. Med. 10:305-309 (2004); Fu A. L., et al. (2004) “Alternative Therapy Of Alzheimer's Disease Via Supplementation With Choline Acetyltransferase,” Neurosci. Lett. 368:258-262; Del Gazio Moore et al. (2004) “Transactivator Of Transcription Fusion Protein Transduction Causes Membrane Inversion,” J. Biol. Chem. 279(31):32541-32544; US Application Publication No. 2007/0105775; Gump et al. (2007) “TAT Transduction: The Molecular Mechanism And Therapeutic Prospects,” Trends in Molecular Medicine, 13(10):443-448; Tilstra, J. et al. (2007) “Protein Transduction: Identification, Characterization And Optimization,” Biochem. Soc. Trans. 35(Pt 4):811-815; WO/2006/121579; US Application Publication No. 2006/0222657). In certain embodiments, a PTD may be covalently cross-linked to an amino acid sequence of the invention or synthesized as a fusion protein with an amino acid sequence of the invention followed by administration of the covalently cross-linked amino acid sequence and the PTD or the fusion protein comprising the amino acid sequence and the PTD. In other embodiments, methods for delivering an amino acid sequence of the invention includes a non-covalent peptide-based method using an amphipathic peptide as disclosed by, for example, Morris, M. C. et al. (2001) “A Peptide Carrier For The Delivery Of Biologically Active Proteins Into Mammalian Cells,” Nat. Biotechnol. 19:1173-1176 and U.S. Pat. No. 6,841,535, and indirect polyethylenimine cationization as disclosed by, for example, Kitazoe et al. (2005) “Protein Transduction Assisted By Polyethylenimine-Cationized Carrier Proteins,” J. Biochem. 137:693-701.

As a non-limiting illustration of a method of making a PTD fusion protein, an expression system that permits the rapid cloning and expression of in-frame fusion polypeptides using an N-terminal 11 amino acid sequence corresponding to amino acids 47-57 of TAT (SEQ ID NO:10 YGRKKRRQRRR) is used (Becker-Hapak, M. et al. (2001) “TAT-Mediated Protein Transduction Into Mammalian Cells,” Methods 24(3):247-56 (2001); Schwarze, F. R. et al. “in vivo Protein Transduction: Delivery Of A Biologically Active Protein Into The Mouse,” (1999) Science 285:1569-72; Becker-Hapak, M. et al. (2003) “Protein Transduction: Generation of Full-Length Transducible Proteins Using the TAT System,” Curr. Protoc. Cell Biol. Chapter 20:Unit 20.2). Using this expression system, cDNA of the amino acid sequence of interest is cloned in-frame with the N-terminal 6× His-TAT-HA (SEQ ID NO:11 HHHHHHYGRKKRRQRRR) encoding region in the pTAT-HA expression vector. The 6× His (SEQ ID NO: 12: HHHHHH) motif provides for the convenient purification of a fusion polypeptide using metal affinity chromatography and the HA epitope tag allows for immunological analysis of the fusion polypeptide. Although recombinant polypeptides can be expressed as soluble proteins within E. coli, TAT-fusion polypeptides are often found within bacterial inclusion bodies. In the latter case, these proteins are extracted from purified inclusion bodies in a relatively pure form by lysis in denaturant, such as, for example, 8 M urea. The denaturation aids in the solubilization of the recombinant polypeptide and assists in the unfolding of complex tertiary protein structure which has been observed to lead to an increase in the transduction efficiency over highly-folded, native proteins (Becker-Hapak, M. et al. (2001) “TAT-Mediated Protein Transduction Into Mammalian Cells,” Methods 24(3):247-56 (2001)). This latter observation is in keeping with earlier findings that supported a role for protein unfolding in the increased cellular uptake of the TAT-fusion polypeptide TAT-DHFR (Bonifaci, N. et al. (1995) “Nuclear Translocation Of An Exogenous Fusion Protein Containing HIV Tat Requires Unfolding,” Aids 9:995-1000). It is thought that the higher energy of partial or fully denatured proteins may transduce more efficiently than lower energy, correctly folded proteins, in part due to increased exposure of the TAT domain. Once inside the cells, these denatured proteins are properly folded by cellular chaperones such as, for example, HSP90 (Schneider, C. et al. (1996) “Pharmacologic Shifting Of A Balance Between Protein Refolding And Degradation Mediated By Hsp90,” Proc. Natl. Acad. Sci. (U.S.A.) 93(25): 14536-14541 (1996)). Following solubilization, bacterial lysates are incubated with NiNTA resin (Qiagen), which binds to the 6× His domain in the recombinant protein. After washing, proteins are eluted from the column using increasing concentrations of imidazole. Proteins are further purified using ion exchange chromatography and finally exchanged into PBS +10% glycerol by gel filtration.

In certain embodiments the invention encompasses administration of an amino acid sequence of the invention conjugated to, fused with, or otherwise combined with, a PTD. In other embodiments, the invention encompasses administration of a nucleic acid sequence of the invention conjugated to, fused with, or otherwise combined with, a PTD. Both, an amino acid sequence and a nucleic acid sequence can be transduced across a cellular membrane when conjugated to, fused with, or otherwise combined with, a PTD. As such, administration of an amino acid sequence and a nucleic acid sequence is encompassed by the present invention. Routes of administration of an amino acid sequence or nucleic acid sequence of the invention include, for example, intraarterial administration, epicutaneous administration, ocular administration (e.g., eye drops), intranasal administration, intragastric administration (e.g., gastric tube), intracardiac administration, subcutaneous administration, intraosseous infusion, intrathecal administration, transmucosal administration, epidural administration, insufflation, oral administration (e.g., buccal or sublingual administration), oral ingestion, anal administration, inhalation administration (e.g., via aerosol), intraperitoneal administration, intravenous administration, transdermal administration, intradermal administration, subdermal administration, intramuscular administration, intrauterine administration, vaginal administration, administration into a body cavity, surgical administration (e.g., at the location of a tumor or internal injury), administration into the lumen or parenchyma of an organ, or other topical, enteral, mucosal, or parenteral administration, or other method, or any combination of the forgoing as would be known to one of ordinary skill in the art (see, for example, REMINGTON'S PHARMACEUTICAL SCIENCES, 18th Ed. Mack Printing Company, 1990, incorporated herein by reference).

Having now generally described the invention, the same will be more readily understood through reference to the following examples, which are provided by way of illustration and are not intended to be limiting of the present invention unless specified.

### EXAMPLE 1

### Materials and Methods

### Molecular Biology

The enhanced cyan fluorescent protein (eCFP) and Citrine clones are described by (Griesbeck, O. et al. (2001) “Reducing The Environmental Sensitivity Of Yellow Fluorescent Protein. Mechanism And Applications,” J. Biol. Chem. 276:29188-29194; Miyawaki, A. et al. (1997) “Fluorescent Indicators For Ca²⁺ Based On Green Fluorescent Proteins And Calmodulin,” Nature 388:882-887). HERG channels fused to fluorescent proteins, site-directed point mutations and deletion mutations were made using an overlapping PCR strategy and confirmed with DNA sequencing. HERG channel cDNA's were subcloned into a modified pGEMHE vector for heterologous expression. RNA was transcribed with the mESSAGE mACHINE kit (Ambion, Austin, Tex.) and injected with a micropipette into Xenopus oocytes. Oocytes were prepared as described elsewhere (Herzberg, I. M. et al. (1998) “Transfer Of Rapid Inactivation And E-4031 Sensitivity From HERG to M-EAG Channels,” J. Physiol. 511(1):3-14) and incubated for 3-20 days at 16° C.

### Electrophysiology

Ionic currents from HERG channels expressed in oocytes were recorded with an OC-725C two-electrode voltage clamp (Warner Instruments). Data were digitized with an ITC-18 (Instrutech, Great Neck, N.Y.) and recorded and analyzed with the PatchMaster software package (Instrutech) and Igor software (Lake Oswego, Oreg.) running on a Pentium 4 computer. The electrodes contained 2M KCl. The bath (external) solution contained 94 mM NaCl, 4 mM KCl, 1 mM MgCl₂ and 0.3 mM CaCl₂, pH 7.4.

Ionic currents from I_{Kr} channels in guinea pig myocytes are recorded with the HEKA 10 patch-clamp ephysiol. Methods for recording and analyzing I_{Kr} from adult guinea pig myocytes may be adapted from the descriptions provided by Weerapura, M. et al. (2002) (“A Comparison Of Currents Carried By HERG, With And Without Coexpression Of MiRP1, And The Native Rapid Delayed Rectifier Current. Is MiRP1 The Missing Link?” J. Physiol. (Lond) 540:15-27). Currents are recorded in the whole-cell mode. To record action potentials, the current-clamp mode is employed. To separate I_{Kr} from other voltage dependent currents, nimodipene (1 μM) is used to block I_{Ca}, and chromanol 293B (50 μM) is used to block I_{Ks}. These compounds do not inhibit I_{Kr}. A holding potential of −40 is used to inactivate I_{Na}. Temperature is controlled at 35-37° C. Solutions are applied with a rapid solution changer (RSC-160). At the conclusion of experiments, I_{Kr} is verified by E-4031 inhibition. External solution is 145 mM NaCl, 5 mM KCl, 1.8 mM CaCl₂, 1 mM MgCl₂, 10 mM HEPES, pH 7.4. The internal (patch pipette) solution is 140 KCl, 5 mM K₂ATP, 5 MgCl₂, 5 mM EGTA and 10 HEPES, pH 7.2.

### Adenoviral Transfer

Recombinant adenoviruses expressing HERG proteins are produced for reliable, high-efficiency delivery into adult cardiac myocytes cells. Polynucleotides encoding the HERG N-Terminal Domain (or the negative control HERG1a Y43A N-Terminal Domain) are introduced into the pShuttle-CMV vector and recombinant adenoviral plasmids generated (He, T. C. et al. (1998) “A Simplified System For Generating Recombinant Adenoviruses,” Proc. Natl. Acad. Sci. (USA) 95:2509-2514). At a multiplicity of infection of 1-10 plague-forming units per cell, infection of over 80% of cells is obtained without any deleterious effects on cell integrity.

### Confocal Microscopy

Fluorescence emission intensity from whole oocytes expressing fluorescently-labeled channel subunits were collected using a confocal microscope (Zeiss 510 Meta) with laser excitation. The microscope objective was 5× with 0.15 NA or 10× with 0.3 NA. For myocytes emission spectra were obtained with an oil immersion 63× objective. Fluorescence data were acquired and analyzed with the MetaMorph software package (Universal Imaging).

### Thermodynamic Mutant Cycle Analysis

Thermodynamic Mutant Cycle analysis is employed to test for specific interactions between amino acids on interacting surfaces (Craven, K. B. et al. (2004) “Salt Bridges and Gating in the COOH-terminal Region of HCN2 and CNGA1 Channels,” J. Gen. Physiol. 124:663-677; Finlayson, K. et al. (2004) “Acquired QT Interval Prolongation And HERG: Implications For Drug Discovery And Development,” Eur. J. Pharmacol. 500:129-142). Thermodynamic Mutant Cycle analysis is used to separate allosteric or indirect effects of mutations from direct interaction effects due to an interaction between the mutated residues. Thermodynamic Mutant Cycle analysis has been used to study the interactions between amino acids in proteins (Carter, P. J. et al. (1984) “The Use Of Double Mutants To Detect Structural Changes In The Active Site Of The Tyrosyl-tRNA synthetase (Bacillus stearothermophilus),” Cell 38:835-840), the interaction between a toxin and a potassium channel (Hidalgo, P. et al. (1995) “Revealing The Architecture Of A K⁺ Channel Pore Through Mutant Cycles With A Peptide Inhibitor,” Science 268:307-310), a cyclic nucleotide gated-channel and cyclic nucleotides (Sunderman, E. R. et al. (1999) “Mechanism of allosteric modulation of rod cyclic nucleotide-gated channels,” J. Gen. Physiol 113:601-620). Coupling energies greater than 1 kcal/mol are generally the criteria for a direct interaction between two molecules. To carry out Thermodynamic Mutant Cycle, the change in free energy ΔG=−RT (1 n K) is calculated based on a simplified model for closing in which channels go from the open (O) state to the closed (C) state at negative voltages (Scheme 2).

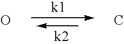

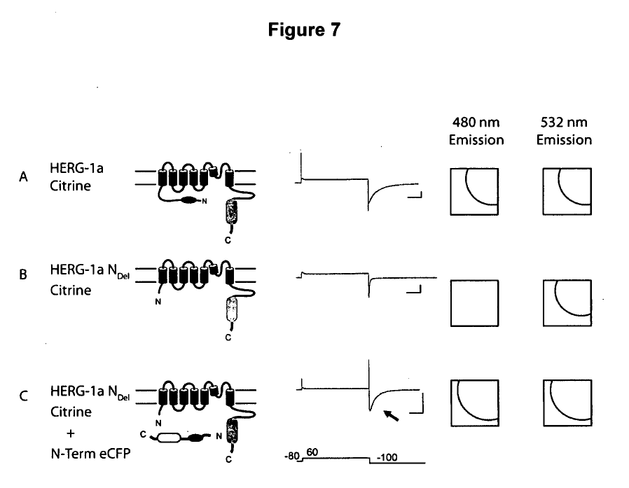

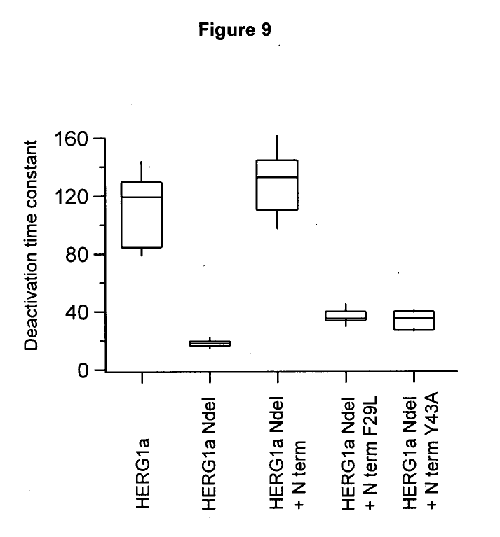

In this model, the k1 and k2 are the rates for the forward (O to C) and reverse (C to O) transitions, and the equilibrium constant K=k2/k1. In HERG, since the O to C transition is dominant at very negative voltages (FIG. 7 and FIG. 9), k2 is small. Thus τ=1/k1+k2 can be estimated as 1/k1 and k1 is equal to 1/K. This same analysis has been used elsewhere for HERG (Wang, J. et al. (2000) “Dynamic Control Of Deactivation Gating By A Soluble Amino-Terminal Domain In HERG K(⁺) Channels,” J. Gen. Physiol. 115:749-758; Wang, J. et al. (1998) “Regulation Of Deactivation By An Amino Terminal Domain In Human Ether-A-Go-Go-Related Gene Potassium Channels,” J. Gen. Physiol. 112:637-647 [published erratum appears in J. Gen. Physiol. 113(2):359 (1999)]). The free energy change ΔG=−RT 1 n K (where R is the gas constant and T is the temperature) for the transition is calculated. In this way, the free energy for the deactivation transition for each channel bearing mutations in the PAS-CAP region is quantified.

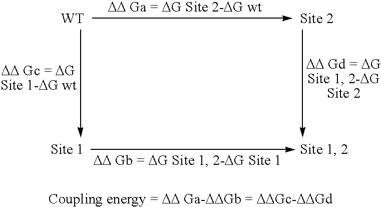

### EXAMPLE 2

### HERG-1a N-Terminal Domain is Capable of Rescuing Slow Deactivation Gating in HERG Channels

A polynucleotide (SEQ ID NO:13) encoding the “HERG-1a N-Terminal Domain” (i.e. the first 135 amino acids of HERG-1a, including the PAS domain and the PAS-CAP region) was fused with a polynucleotide (SEQ ID NO:14) encoding the cyan fluorescent protein (ECFP) (SEQ ID NO:15) to encode a fusion protein in which the ECFP is fused to the carboxy terminus of the HERG-1a N-Terminal Domain.

SEQ ID NO: 13

ATGCCGGTGC GGAGGGGCCA CGTCGCGCCG CAGAACACCT
TCCTGGACAC CATCATCCGC AAGTTTGAGG GCCAGAGCCG
TAAGTTCATC ATCGCCAACG CTCGGGTGGA GAACTGCGCC
GTCATCTACT GCAACGACGG CTTCTGCGAG CTGTGCGGCT
ACTCGCGGGC CGAGGTGATG CAGCGACCCT GCACCTGCGA
CTTCCTGCAC GGGCCGCGCA CGCAGCGCCG CGCTGCCGCG
CAGATCGCGC AGGCACTGCT GGGCGCCGAG GAGCGCAAAG
TGGAAATCGC CTTCTACCGG AAAGATGGGA GCTGCTTCCT
ATGTCTGGTG GATGTGGTGC CCGTGAAGAA CGAGGATGGG
GCTGTCATCA TGTTCATCCT CAATTTCGAA GTGGTGATGG AGAAG

SEQ ID NO: 14

ATGGTGAGCA AGGGCGAGGA GCTGTTCACC GGGGTGGTGC
CCATCCTGGT CGAGCTGGAC GGCGACGTAA ACGGCCACAG
GTTCAGCGTG TCCGGCGAGG GCGAGGGCGA TGCCACCTAC
GGCAAGCTGA CCCTGAAGTT CATCTGCACC ACCGGCAAGC
TGCCCGTGCC CTGGCCCACC CTCGTGACCA CCCTGACCTG
GGGCGTGCAG TGCTTCAGCC GCTACCCCGA CCACATGAAG
CAGCACGACT TCTTCAAGTC CGCCATGCCC GAAGGCTACG
TCCAGGAGCG TACCATCTTC TTCAAGGACG ACGGCAACTA
CAAGACCCGC GCCGAGGTGA AGTTCGAGGG CGACACCCTG
GTGAACCGCA TCGAGCTGAA GGGCATCGAC TTCAAGGAGG
ACGGCAACAT CCTGGGGCAC AAGCTGGAGT ACAACTACAT
CAGCCACAAC GTCTATATCA CCGCCGACAA GCAGAAGAAC
GGCATCAAGG CCCACTTCAA GATCCGCCAC AACATCGAGG
ACGGCAGCGT GCAGCTCGCC GACCACTACC AGCAGAACAC
CCCCATCGGC GACGGCCCCG TGCTGCTGCC CGACAACCAC
TACCTGAGCA CCCAGTCCAA GCTGAGCAAA GACCCCAACG
AGAAGCGCGA TCACATGGTC CTGCTGGAGT TCGTGACCGC
CGCCGGGATC ACTCTCGGCA TGGACGAGCT GTACAAGTAA

SEQ ID NO: 15

MVSKGEELFT GVVPILVELD GDVNGHRFSV SGEGEGDATY
GKLTLKFICT TGKLPVPWPT LVTTLTWGVQ CFSRYPDHMK
QHDFFKSAMP EGYVQERTIF FKDDGNYKTR AEVKFEGDTL
VNRIELKGID FKEDGNILGH KLEYNYISHN VYITADKQKN
GIKAHFKIRH NIEDGSVQLA DHYQQNTPIG DGPVLLPDNH
YLSTQSKLSK DPNEKRDHMV LLEFVTAAGI TLGMDELYK

The fusion polynucleotide was cloned into the Xenopus expression vector pGH19 (Robertson, G. A. et al. (1996) “Potassium Currents Expressed From Drosophila And Mouse Eag Cdnas In Xenopus Oocytes,” Neuropharmacol. 35:841-850) and introduced into Xenopus cells in concert with a polynucleotide that encodes citrine (a fluorescent protein; SEQ ID NO:16, encoded by SEQ ID NO:17) fused to the carboxy terminus of a HERG-1a variant that lacks residues 2-354 of the N-terminal region (HERG N_{Del}) (SEQ ID NO:18, encoded by SEQ ID NO:19).

SEQ ID NO: 16

MVSKGEELFT GVVPILVELD GDVNGHKFSV SGEGEGDATY
GKLTLKFICT TGKLPVPWPT LVTTFGYGLM CFARYPDHMK
QHDFFKSAMP EGYVQERTIF FKDDGNYKTR AEVKFEGDTL
VNRIELKGID FKEDGNILGH KLEYNYNSHN VYIMADKQKN
GIKVNFKIRH NIEDGSVQLA DHYQQNTPIG DGPVLLPDNH
YLSYQSKLSK DPNEKRDHMV LLEFVTAAGI TLGMDELYK

SEQ ID NO: 17

ATGGTGAGCA AGGGCGAGGA GCTGTTCACC GGGGTGGTGC
CCATCCTGGT CGAGCTGGAC GGCGACGTAA ACGGCCACAA
GTTCAGCGTG TCCGGCGAGG GCGAGGGCGA TGCCACCTAC
GGCAAGCTGA CCCTGAAGTT CATCTGCACC ACCGGCAAGC
TGCCCGTGCC CTGGCCCACC CTCGTGACCA CCTTCGGCTA
CGGCCTGATG TGCTTCGCCC GCTACCCCGA CCACATGAAG
CAGCACGACT TCTTCAAGTC CGCCATGCCC GAAGGCTACG
TCCAGGAGCG CACCATCTTC TTCAAGGACG ACGGCAACTA
CAAGACCCGC GCCGAGGTGA AGTTCGAGGG CGACACCCTG
GTGAACCGCA TCGAGCTGAA GGGCATCGAC TTCAAGGAGG
ACGGCAACAT CCTGGGGCAC AAGCTGGAGT ACAACTACAA
CAGCCACAAC GTCTATATCA TGGCCGACAA GCAGAAGAAC
GGCATCAAGG TGAACTTCAA GATCCGCCAC AACATCGAGG
ACGGCAGCGT GCAGCTCGCC GACCACTACC AGCAGAACAC
CCCCATCGGC GACGGCCCCG TGCTGCTGCC CGACAACCAC
TACCTGAGCT ACCAGTCCAA GCTGAGCAAA GACCCCAACG
AGAAGCGCGA TCACATGGTC CTGCTGGAGT TCGTGACCGC
CGCCGGGATC ACTCTCGGCA TGGACGAGCT GTACAAGTAA

SEQ ID NO: 18

ATGGACCGTG AGATCATAGC ACCTAAGATA AAGGAGCGAA
CCCACAATGT CACTGAGAAG GTCACCCAGG TCCTGTCCCT
GGGCGCCGAC GTGCTGCCTG AGTACAAGCT GCAGGCACCG
CGCATCCACC GCTGGACCAT CCTGCATTAC AGCCCCTTCA
AGGCCGTGTG GGACTGGCTC ATCCTGCTGC TGGTCATCTA
CACGGCTGTC TTCACACCCT ACTCGGCTGC CTTCCTGCTG
AAGGAGACGG AAGAAGGCCC GCCTGCTACC GAGTGTGGCT
ACGCCTGCCA GCCGCTGGCT GTGGTGGACC TCATCGTGGA
CATCATGTTC ATTGTGGACA TCCTCATCAA CTTCCGCACC
ACCTACGTCA ATGCCAACGA GGAGGTGGTC AGCCACCCCG
GCCGCATCGC CGTCCACTAC TTCAAGGGCT GGTTCCTCAT
CGACATGGTG GCCGCCATCC CCTTCGACCT GCTCATCTTC
GGCTCTGGCT CTGAGGAGCT GATCGGGCTG CTGAAGACTG
CGCGGCTGCT GCGGCTGGTG CGCGTGGCGC GGAAGCTGGA
TCGCTACTCA GAGTACGGCG CGGCCGTGCT GTTCTTGCTC
ATGTGCACCT TTGCGCTCAT CGCGCACTGG CTAGCCTGCA
TCTGGTACGC CATCGGCAAC ATGGAGCAGC CACACATGGA
CTCACGCATC GGCTGGCTGC ACAACCTGGG CGACCAGATA
GGCAAACCCT ACAACAGCAG CGGCCTGGGC GGCCCCTCCA
TCAAGGACAA GTATGTGACG GCGCTCTACT TCACCTTCAG
CAGCCTCACC AGTGTGGGCT TCGGCAACGT CTCTCCCAAC
ACCAACTCAG AGAAGATCTT CTCCATCTGC GTCATGCTCA
TTGGCTCCCT CATGTATGCT AGCATCTTCG GCAACGTGTC
GGCCATCATC CAGCGGCTGT ACTCGGGCAC AGCCCGCTAC
CACACACAGA TGCTGCGGGT GCGGGAGTTC ATCCGCTTCC
ACCAGATCCC CAATCCCCTG CGCCAGCGCC TCGAGGAGTA
CTTCCAGCAC GCCTGGTCCT ACACCAACGG CATCGACATG
AACGCGGTGC TGAAGGGCTT CCCTGAGTGC CTGCAGGCTG
ACATCTGCCT GCACCTGAAC CGCTCACTGC TGCAGCACTG
CAAACCCTTC CGAGGGGCCA CCAAGGGCTG CCTTCGGGCC
CTGGCCATGA AGTTCAAGAC CACACATGCA CCGCCAGGGG
ACACACTGGT GCATGCTGGG GACCTGCTCA CCGCCCTGTA
CTTCATCTCC CGGGGCTCCA TCGAGATCCT GCGGGGCGAC
GTCGTCGTGG CCATCCTGGG GAAGAATGAC ATCTTTGGGG
AGCCTCTGAA CCTGTATGCA AGGCCTGGCA AGTCGAACGG
GGATGTGCGG GCCCTCACCT ACTGTGACCT ACACAAGATC
CATCGGGACG ACCTGCTGGA GGTGCTGGAC ATGTACCCTG
AGTTCTCCGA CCACTTCTGG TCCAGCCTGG AGATCACCTT
CAACCTGCGA GATACCAACA TGATCCCGGG CTCCCCCGGC
AGTACGGAGT TAGAGGGTGG CTTCAGTCGG CAACGCAAGC
GCAAGTTGTC CTTCCGCAGG CGCACGGACA AGGACACGGA
GCAGCCAGGG GAGGTGTCGG CCTTGGGGCC GGGCCGGGCG
GGGGCAGGGC CGAGTAGCCG GGGCCGGCCG GGGGGGCCGT
GGGGGGAGAG CCCGTCCAGT GGCCCCTCCA GCCCTGAGAG
CAGTGAGGAT GAGGGCCCAG GCCGCAGCTC CAGCCCCCTC
CGCCTGGTGC CCTTCTCCAG CCCCAGGCCC CCCGGAGAGC
CGCCGGGTGG GGAGCCCCTG ATGGAGGACT GCGAGAAGAG
CAGCGACACT TGCAACCCCC TGTCAGGCGC CTTCTCAGGA
GTGTCCAACA TTTTCAGCTT CTGGGGGGAC AGTCGGGGCC
GCCAGTACCA GGAGCTCCCT CGATGCCCCG CCCCCACCCC
CAGCCTCCTC AACATCCCCC TCTCCAGCCC GGGTCGGCGG
CCCCGGGGCG ACGTGGAGAG CAGGCTGGAT GCCCTCCAGC
GCCAGCTCAA CAGGCTGGAG ACCCGGCTGA GTGCAGACAT
GGCCACTGTC CTGCAGCTGC TACAGAGGCA GATGACGCTG
GTCCCGCCCG CCTACAGTGC TGTGACCACC CCGGGGCCTG
GCCCCACTTC CACATCCCCG CTGTTGCCCG TCAGCCCCCT
CCCCACCCTC ACCTTGGACT CGCTTTCTCA GGTTTCCCAG
TTCATGGCGT GTGAGGAGCT GCCCCCGGGG GCCCCAGAGC
TTCCCCAAGA AGGCCCCACA CGACGCCTCT CCCTACCGGG
CCAGCTGGGG GCCCTCACCT CCCAGCCCCT GCACAGACAC
GGCTCGGACC CGGGCAGT

SEQ ID NO: 19

MREIIAPKIK ERTHNVTEKV TQVLSLGADV LPEYKLQAPR
IHRWTILHYS PFKAVWDWLI LLLVIYTAVF TPYSAAFLLK
ETEEGPPATE CGYACQPLAV VDLIVDIMFI VDILINFRTT
YVNANEEVVS HPGRIAVHYF KGWFLIDMVA AIPFDLLIFG
SGSEELIGLL KTARLLRLVR VARKLDRYSE YGAAVLFLLM
CTFALIAHWL ACIWYAIGNM EQPHMDSRIG WLHNLGDQIG
KPYNSSGLGG PSIKDKYVTA LYFTFSSLTS VGFGNVSPNT
NSEKIFSICV MLIGSLMYAS IFGNVSAIIQ RLYSGTARYH
TQMLRVREFI RFHQIPNPLR QRLEEYFQHA WSYTNGIDMN
AVLKGFPECL QADICLHLNR SLLQHCKPFR GATKGCLRAL
AMKFKTTHAP PGDTLVHAGD LLTALYFISR GSIEILRGDV
VVAILGKNDI FGEPLNLYAR PGKSNGDVRA LTYCDLHKIH
RDDLLEVLDM YPEFSDHFWS SLEITFNLRD TNMIPGSPGS
TELEGGFSRQ RKRKLSFRRR TDKDTEQPGE VSALGPGRAG
AGPSSRGRPG GPWGESPSSG PSSPESSEDE GPGRSSSPLR
LVPFSSPRPP GEPPGGEPLM EDCEKSSDTC NPLSGAFSGV
SNIFSFWGDS RGRQYQELPR CPAPTPSLLN IPLSSPGRRP
RGDVESRLDA LQRQLNRLET RLSADMATVL QLLQRQMTLV
PPAYSAVTTP GPGPTSTSPL LPVSPLPTLT LDSLSQVSQF
MACEELPEGA PELPQEGPTR RLSLPGQLGA LTSQPLHRHG SDPGS

In channels lacking the N-terminal region (HERG N_{Del}), deactivation was fast, as expected (FIG. 7B). When the HERG-1a N-Terminal Domain was co-expressed with HERG N_{Del}, the resulting channels are found to exhibited slow deactivation gating (FIG. 7C), like HERG-1a channels (FIG. 7A). This result demonstrates a polynucleotide encoding amino acid residues of the “HERG-1a N-Terminal domain” is capable of rescuing slow deactivation gating in HERG channels.

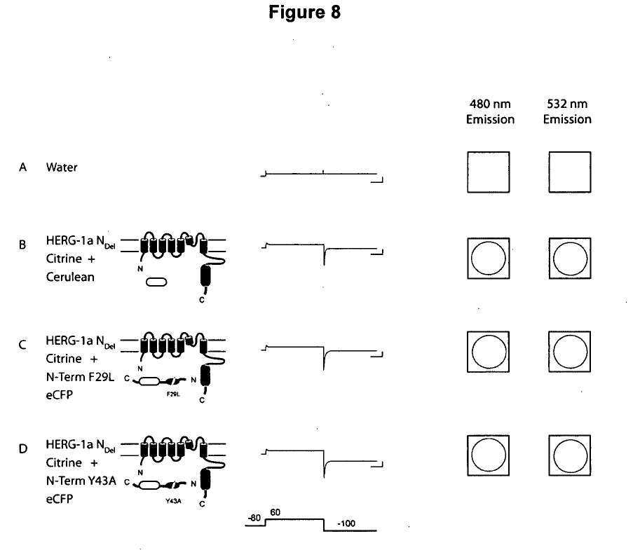

To test the specificity of the HERG-1a N-Terminal Domain, mutations F29L or Y43A were made. These mutations have been shown to speed deactivation in intact HERG channels. HERG-1a N-terminal domain bearing these mutations did not rescue slow deactivation gating (FIG. 8, Panel C and Panel D). Since these fragments were genetically fused to eCFP, experiments were conducted to determine if reduced expression of the HERG-1a N-Terminal Domain was the cause of the observed lack of rescue of function. Such investigation revealed that HERG-1a N-Terminal Domain variants having F29L or Y43A mutations had a robust fluorescence signal, like the wild-type HERG-1a N-Terminal Domain, thus indicating robust protein expression. The specificity of the HERG-1a N-Terminal Domain was further confirmed in control experiments with soluble eCerulean cyan fluorescent protein (eCPF). eCPF was co-expressed with HERG N_{Del} channels. Soluble eCerulean was highly expressed in cells, but, as anticipated, did not alter deactivation kinetics (FIG. 8, Panel B). Thus, the F29 and Y43 sites in the fragment, as in intact channels, are critical for slow deactivation. Together, the results show that the HERG-1a N-Terminal Domain is a specific mediator of slow deactivation. Importantly, the HERG-1a N-Terminal Domain restored deactivation to the same rate as seen in wild-type channels (FIG. 8). Thus, this region contains the necessary determinants for slow deactivation gating.

### EXAMPLE 3

### The Soluble HERG-1a N-Terminal Domain Confers Slow Deactivation Gating to HERG-1b Channels

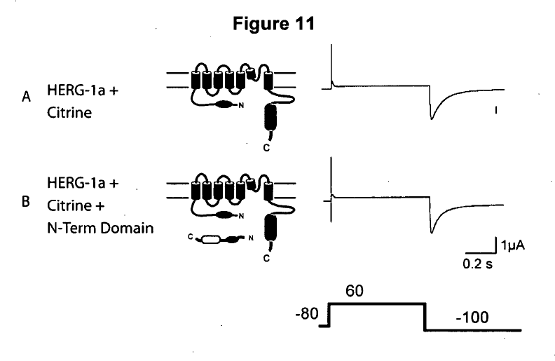

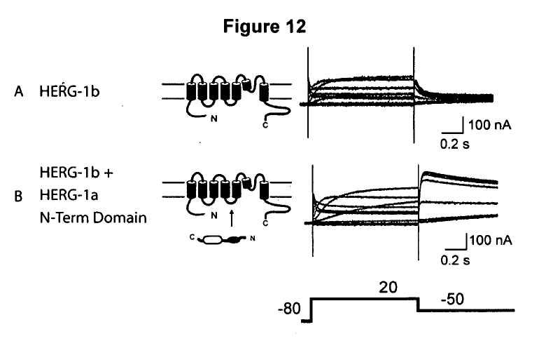

To determine if the HERG-1a N-Terminal Domain could target to HERG-1b channels, the HERG-1a N-Terminal Domain was co-expressed with HERG-1b channels. As a control, HERG-1a was expressed alone or with the HERG-1a N-terminal domain. As shown in FIG. 11, Panels A and B, the N-terminal domain did not change wild-type deactivation kinetic of HERG1a in these channels. HERG-1b channels expressed alone exhibited fast deactivation kinetics, as expected (FIG. 12, Panel A). However, when HERG-1b was co-expressed with the HERG-1a N-Terminal Domain, the deactivation kinetics were about 10-fold slower than for wild-type HERG01b currents (FIG. 12, Panel B). Thus, the HERG-1a N-Terminal Domain confers slow deactivation to the HERG-1b variant, and the HERG-1a N-Terminal Domain is a specific, functional probe of HERG-1b subunits.

### EXAMPLE 4

### Adenoviral Transfer of Fluorescent Fusion Proteins to Native Myocytes

One aspect of the present invention relates to the ability to rescue genetic or acquired HERG deficiencies by providing native myocytes with expressible polynucleotides encoding the relevant portions of the HERG-1a N-Terminal Domain. To demonstrate this aspect of the invention, A Kir 1.2 channel fused to GFP was transferred to native adult cardiomyocytes. Adenoviral-mediated transfer was employed to infect native myocytes with a polynucleotide encoding Kir 2.1-GFP. Confocal imaging of the myocyte shows fluorescence intensity throughout the cell. This shows that K channels fused to FP's can be successfully transferred to native cells and imaged.

### EXAMPLE 5

### Determination of the Functional Significance for Deactivation Gating of Negatively Charged Residues in the PAS-CAP Region in the N-Terminal Domain of the HERG Channel

A PAS-CAP region in rEag channels interacted with the H343 residue in the channel S4 (Terlau, H. et al. (1997) “Amino Terminal-Dependent Gating Of The Potassium Channel Rat eag Is Compensated By A Mutation In The S4 Segment,” J. Physiol. (Lond) 502:537-543). Furthermore, mutations at charged residues in the lower S4 region of HERG (e.g., D540 and E544) sped up the deactivation kinetics of HERG ; Tristani-Firouzi, M. et al. (2002) “Interactions between S4-S5 linker and S6 Transmembrane Domain Modulate Gating Of HERG K⁺ Channels,” J. Biol. Chem. 277:18994-9000). The invention exploits these findings to identify sites that interact with the HERG PAS-CAP. To accomplish this goal, a series of mutations at the base of the HERG voltage-sensor region are made. Tests are then conducted for determinants in the HERG lower S4 region at charged sites that flank the equivalent H343 residue in Eag. This experiment is in three parts: i) point mutations are made in the lower S4 region at negatively charged sites. These include the D540 and E544 in HERG. Replacement residues are selected so as to reverse the charge at these sites (e.g., D540R and D540K and E544R and E544K). These four mutant channels are expressed and characterized. Since they differ from sites in Eag, and since H343 plays a key role in Eag activation, residues at those sites in HERG will be mutated (e.g., R541H and S5431). Charge reversal mutations are also made to residues in the PAS-CAP region (e.g., R4E, R4D, R5E, R5D and H7E, H7D). These channels are expressed and individually characterized. Channels are then constructed bearing two charge reversals, one in the positive to negative in the PAS-CAP region plus negative to positive in the lower S4 (e.g., HERG-1a having the mutations R4D and D540R). The deactivation gating of these channels is determined with exponential fits to the deactivation phase.

The experiments reveal: i) that channels bearing negatively charged side-chains in the PAS-CAP have fast deactivation kinetics (based on the fast kinetics of the triple mutation in the PAS-CAP region); ii) that channels with mutations in the lower S4 will exhibit fast deactivation as described in other studies (Sanguinetti, M. C. et al. (1999) “Mutations Of The S4-S5 Linker Alter Activation Properties Of HERG Potassium Channels Expressed In Xenopus Oocytes,” J. Physiol. (Lond) 514:667-675); and iii) that HERG channels bearing complementary charge reversals in the PAS-CAP and lower S4 (e.g., HERG R4D, D540R) result in slow deactivation. To quantify putative interactions between PAS-CAP residues and lower S4 residues, Thermodynamic Mutant Cycle analysis is employed (see above). Thermodynamic Mutant Cycle analysis distinguishes between indirect effects due to allostery and direct effects due to a physical interaction between two residues by comparing the free energy of the interactions as measured above. The results indicate that key sites in the PAS-CAP and lower S4 region are necessary for slow deactivation gating, and that the PAS-CAP and lower S4 form a direct positive-negative salt bridge that is required for slow deactivation gating. The Thermodynamic Mutant Cycle analysis quantifies this interaction with a measure of the free energy of coupling between sites that interact directly. Taken together, disruption of slow deactivation by individual mutations in the PAS-CAP and individual mutations lower S4 combined with restoration of slow deactivation kinetics in channels with complementary charge reversals indicates an interaction between these regions that is based on electrostatics or salt bridge formation.

An alternative strategy is to make a set of alanine mutations at the additional sites in the lower S4 (for example, HERG R541A) and then introduce these to channels bearing PAS-CAP mutants and use Thermodynamic Mutant Cycle analysis to test for direct interactions as above, however, mutations at some sites may not produce functional channels; thus functional channel formation is monitored in this approach.

### EXAMPLE 6

### Identification of the Intracellular Determinants that Comprise A PAS Receptor Domain

Despite advances in identifying N-terminal regions that are determinants of gating, the regions that the N-terminal regions interact with have not been elucidated. These interactions are significant since they form the basis for slow deactivation and the resurgent current the repolarizes the ventricular action potential. One aspect of the invention relates to the recognition that hydrophobic residues on the surface of the PAS domain interact with hydrophobic intracellular sites that form a PAS receptor. These sites are primarily located in a hydrophobic region previously thought to be part of the channel S5 domain and in sites in the S4-S5 linker and S6, as described below.

As discussed above, the invention reveals that the PAS domain must bind to the channel. The evidence is that: (a) a purified peptide encoding the HERG-1a N-terminal domain interacted with HERG Ndel channels and (b) a similar region encoding the HERG-1a N-terminal domain restored deactivation when introduced as a gene fragment (FIGS. 7, 8, and 9). The invention also reveals that the S4-S5 linker and lower S6 regions partially form a “PAS receptor” since mutagenesis studies of the S4-S5 linker produce changes in the slow deactivation gating, consistent with this region being a determinant of slow deactivation (see, Wang, J. et al. (1998) “Regulation Of Deactivation By An Amino Terminal Domain In Human Ether-A-Go-Go-Related Gene Potassium Channels,” J. Gen. Physiol. 112:637-647 [published erratum appears in J. Gen. Physiol. 113(2):359 (1999)]) and the S4-S5 linker is nearby the S6 in HERG (Ferrer, T. et al. (2006) “The S4-S5 Linker Directly Couples Voltage Sensor Movement To The Activation Gate In The Human Ether-A′-Go-Go-Related Gene (hERG) K⁺ Channel,” J. Biol. Chem. 281:12858-12864). Moreover, the crystal structure of Kv1.2 channels reveal that the S4-S5 linker extends further into the S5 region than had been previously thought (Long, S.B. et al. (2005) “Crystal Structure Of A Mammalian Voltage-Dependent Shaker Family K⁺ Channel,” Science 309:897-903). Mapping HERG residues onto this structure shows that a group of hydrophobic residues, previously described as being in S5 instead reside in the S4-S5 linker (Warmke, J. W. et al. (1994) “A Family Of Potassium Channel Genes Related To Eag In Drosophila And Mammals,” Proc. Natl. Acad. Sci. (USA) 91(8):3438-3442). The Kv1.2 structure also showed that the S4-S5 linker was near the lower part of the S6 transmembrane region (Long, S. B. et al. (2005) “Crystal Structure Of A Mammalian Voltage-Dependent Shaker Family K⁺ Channel,” Science 309:897-903).

A biochemical feature of PAS is that the PAS surface has a hydrophobic region and mutations at hydrophobic sites in PAS disrupt deactivation gating (Morais Cabral, J. H. et al. (1998) “Crystal Structure And Functional Analysis Of The HERG Potassium Channel N Terminus: A Eukaryotic PAS Domain,” Cell 95:649-655) and likewise disrupt the genetically encoded HERG-1a N-terminal domain (FIG. 8 and FIG. 9). In summary, evidence indicates that a hydrophobic receptor site for PAS includes sites previously thought to reside in membrane S5 region (and in the S4-S5 linker and S6). The crystal structure of the Kv1.2 channel makes structure-based mutagenesis in HERG feasible.

The present invention provides a new approach to determine a PAS receptor: i.e., using the N-terminal region (reapplied as a gene fragment) to recover deactivation (FIGS. 7, 8 and 9). This finding allows one to test intracellular regions for determinants of functional interactions with the soluble HERG-1a N-terminal domain. Thermodynamic Mutant Cycle analysis is employed to quantify direct interactions involving: i) channels bearing hydrophobic to alanine mutation in the internal regions will be expressed and characterized, and ii) channels bearing hydrophobic to alanine changes that have been co-expressed with the HERG-1a N-terminal domain.

To do so, site-directed mutations of hydrophobic residues in the lower S5 region of the channel are isolated. These sites are underlined on an alignment of Kv1.2 channels with HERG (Kv11.1) channels and extend from V549 to A561 in HERG:

Exposed to Cytoplasm in Kv1.2 Structure

| | | | |
|---|---|---|---|
| Kv1.2 | 307- | LSRHSKGLQILGQTLKASMRE | SEQ ID NO: 20 |
| HERG | 541- | RYSEYGAAVLFLLMCTFALIA | SEQ ID NO: 21 |
| | | Previously Identified as | |
| | | S5 Transmembrane in HERG | |

Residues 540-548 of SEQ ID NO:1 constitute the S4-S5 linker; the lower S6 contains residues 662-667 of SEQ ID NO:1). Site-directed mutations are made these sites to change the native residue to alanine. Preferably, experiments are conducted to form four sets of alanine replacements in triplets (e.g., D540A-R541A-Y542A, etc.). Triplet changes that affect the interaction with the PAS domain are identified and single replacements are made to pinpoint the site of interaction. The mutations are preferably made in the background of the HERG-1a Ndel-S620T-Citrine channel, since this background channel has the advantages of no or very weak inactivation. Thus it is much easier to characterize the effects on the channel of subsequent mutations. The removal of inactivation has no measurable effect on the kinetics of deactivation (Wang, J. et al. (1998) “Regulation Of Deactivation By An Amino Terminal Domain In Human Ether-A-Go-Go-Related Gene Potassium Channels,” J. Gen. Physiol. 112:637-647 [published erratum appears in J. Gen. Physiol. 113(2):359 (1999)]). The role of key amino acid side-chains is tested by replacing the native group of such amino acids with the small, un-reactive, methyl side-chain of alanine.

Channels bearing mutations at hydrophobic sites that are mutated to alanine are evaluated in Xenopus oocytes in order to characterize their kinetics and record their deactivation kinetics. The kinetics of speeding are quantified with exponential fits to the deactivation kinetics (see, FIG. 9) and the free energy of binding is calculated for each PAS domain-mutant PAS receptor using the equation outlined above.

The experiments indicate that: i) for some channels bearing hydrophobic to alanine changes in the intracellular S5 region will have little or no impact on channel gating as determined by a characterization of the kinetics of these channels, including the activation, deactivation and conductance voltage relationships; and ii) with co-expression of the HERG-1a N-Terminal Domain with channels bearing hydrophobic to alanine mutations, deactivation in some of the channels will not be recovered, while at other sites partial or total recovery is observed. The experiments demonstrate that channels that bear hydrophobic to alanine changes that do not affect other components of gating and lack recovery of deactivation when co-expressed with the HERG-1a N-Terminal Domain identify a hydrophobic site that is a key determinant of a PAS receptor. Channels bearing hydrophobic to alanine changes that do not affect other components of gating and do show recovery of deactivation when co-expressed with the HERG-1a N-Terminal Domain identify a hydrophobic site that is not a key determinant of a PAS receptor. Those channels for which the hydrophobic to alanine change does affect other channel properties, may be a determinant of a PAS receptor. In such cases, the mutation is transferred to the background of a channel that has a point-mutation in the PAS domain (such as Y43A) and uses Thermodynamic Mutant Cycle Analysis to determine direct interactions between the sites. Thus, the invention provides method for identifying key determinant of a PAS receptor.

Alternatively, direct interactions between the HERG-1a N-Terminal Domain-eCFP and the HERG N_{Del} channels are identified through an immunoprecipitation reaction. In such an approach, co-immunoprecipitation is performed (after co-expression of HERG N_{Del} channel with a HERG-1a N-Terminal Domain-eCFP molecule) with a primary antibody directed at the HERG-1a C-terminal region (such as HERG-KA). This Ab is anticipated to co-immunoprecipitate the HERG-1a N-Terminal Domain-eCFP molecule. Primary antibodies to eCFP can then be employed to detect the HERG-1a N-Terminal Domain-eCFP molecule.

### EXAMPLE 7

### Functional Role of HERG-1a Isoforms in the Heart: The Differences in Deactivation Gating Kinetics Between ERG-1a and Cardiac I_{Kr} Channels are Due to the Function of ERG-1b Isoform Subunits in Heart

As discussed above, the native cardiac I_{Kr} channel has significantly faster deactivation kinetics than ERG-1a channels. The difference in deactivation kinetics is due to the presence of ERG-1a isoforms, and in particular to the ERG-1b isoform, in heart cells. ERG-1b has faster closing kinetics than ERG1a and I_{Kr} when expressed in heterologous systems however, as discussed above, while ERG-1b is expressed in heart, it has not previously been determined to explain the relatively faster kinetics of deactivation measured for I_{Kr}.

The finding of the present invention that the HERG-1a N-terminal domain is a genetically encoded, specific probe of HERG-1b function (FIG. 11 and FIG. 12) provides a means for investigating the role of HERG-1b in accelerating the deactivation kinetics of ERG-1a channels. A key advantage of such an approach is that the HERG fragments themselves do not form channels and thus test specific modulation of native I_{Kr}.

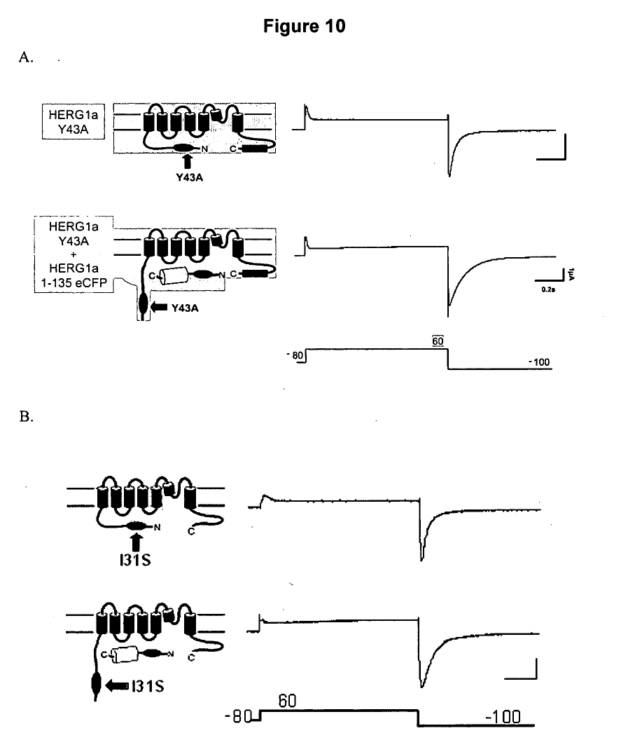

Adenoviral vectors are employed to transfer the genetically-encoded HERG-1a N-Terminal Domain, or a HERG-1a N-terminal domain bearing a mutation that removes the functional slow deactivation gating, i.e. the HERG1a Y43A N-terminal domain (FIG. 8 and FIG. 9) into native myocytes. HERG channels have been successfully transferred to myocytes using adenovirus in other studies (Hoppe, U. C., et al. (2001) “Distinct gene-specific mechanisms of arrhythmia revealed by cardiac gene transfer of two long QT disease genes, HERG and KCNE1,” Proc. Natl. Acad. Sci. (USA) 98:5335-5340; Teng G. Z. X. et al. (2004) “Prolonged Repolarization And Triggered Activity Induced By Adenoviral Expression Of HERG N629D In Cardiomyocytes Derived From Stem Cells,” Cardiovasc. Res. 61 :268-277). Fluorescence confocal microscopy is employed to detect and localize the introduced HERG-1a N-Terminal Domain and the I_{Kr} current is recorded from dissociated adult myocytes bearing the introduced HERG-1a N-Terminal Domain. Additional deactivation experiments to determine that gene transfer experiments can produce measurable changes in deactivation kinetics are conducted in which HERG1a channels that are expected to speed deactivation kinetics in native cells (e.g. HERG1a Y43A and I31S, which expresses functional channels with fast deactivation) are introduced into myocytes and their impact on deactivation kinetics confirmed and the ability of the N-term region to resue function determined (FIG. 10).

The amino terminal domain of HERG1a was co-expressed with HERG channels with mutations in the PAS domain. Mutations are made in HERG (for example, isoleucine 31 to serine [I31S mutation] and tyrosine 43 to alanine [Y43A mutation]). HERG I31S mutation is associated with long QT syndrome. HERG I31S or HERG Y43A channels had fast deactivation kinetics, as anticipated (FIG. 10). Co-expression of HERG I31S or HERG Y43A with the amino terminal domain of HERG1a resulted in channels with slower deactivation kinetics (FIG. 10). The results show that the amino terminal domain of HERG1a restored the slow deactivation kinetics of channels with point mutation in the PAS domain. Because HERG I31S is associated with long QT syndrome and the results show that the N-term region reverses or ameliorates the dysfunctional kinetics of HERG I31S, the amino terminal domain of HERG1a represents a viable therapeutic option for treating long QT syndrome.

### A. Imaging Fixed Myocytes to Determine and Resolve the Expression of the Fragment

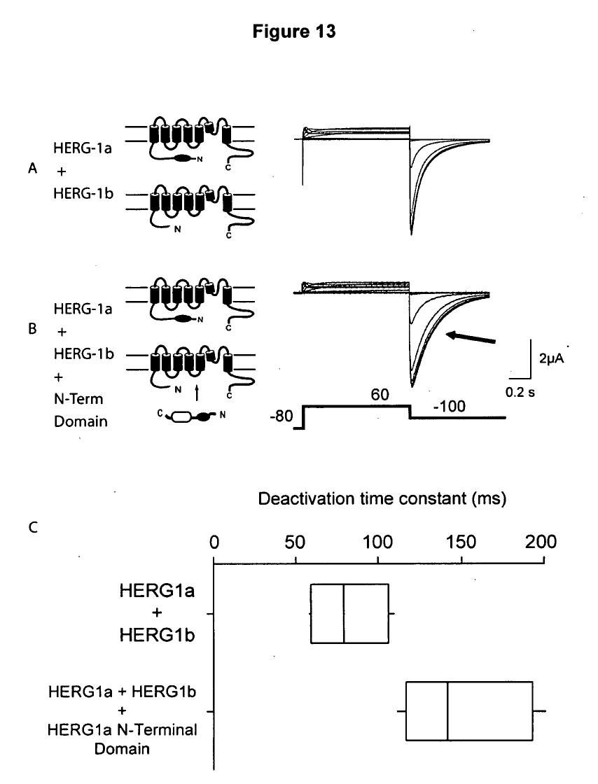

The HERG-1a N-Terminal Domain is fused to eCFP and exhibits robust fluorescence intensity after expression in oocytes (FIG. 7). To identify myocytes that have incorporated the HERG1a N-terminal domain after adenoviral transfer, a laser-scanning confocal microscope is used to image fluorescence from the HERG-1a N-terminal domain-eCFP. Confocal imaging of myocytes expressing Kir 2.1-GFP, have been localized to the Z line, whereas HERG channels have been shown to cluster at the T-tubules (Jones, E. M. et al. (2004) “Cardiac I_{Kr} Channels Minimally Comprise hERG 1a and 1b subunits,” J. Biol. Chem. 279:44690-44694). Thus, dual color imaging using a secondary antibody conjugated to rhodamine (for staining and detection of the Na/Ca exchanger (as in FIG. 13) is used as a reference in the imaging of the HERG-1a N-Terminal Domain-eCFP construct, thereby providing a determination of its spatial localization within the myocytes.

B. Electrophysiological Recordings from Myocytes

Cardiac I_{Kr} currents are recorded from native guinea pig myocytes using the whole-cell patch-clamp technique. To isolate cardiac I_{Kr}, recordings are made in the presence of specific inhibitors of I_{Ks}. The recordings are employed to measure the kinetics of deactivation with exponential fits to determine the time constants for deactivation. Currents are recorded from myocytes that have been selected based on fluorescence intensity to be positive for expression the HERG-1a N-Terminal Domain. Currents are additionally be recorded from control uninfected myocytes and from control myocytes expressing the negative control HERG-1a Y43A domain. Additionally, kinetics are recorded of expressed control HERG Y43A-eCFP and measure the AP in myoctes in current clamp mode.

C. Electrophysiological Recordings from a HERG-1a Cell Line

HERG-1a channels from a permanently transfected human cell lines are recorded using whole-cell patch-clamp technique (Zhou, Z. et al. (1998) “Properties Of HERG Channels Stably Expressed In HEK 293 Cells Studied At Physiological Temperature,” Biophys. J. 74:230-241). The kinetics of deactivation is measured from HERG-1a channels expressed in the human cell line and measure the deactivation kinetics with exponential fits. The time constants of deactivation from HERG-1a channels in the human cell line are compared with those from native myoctyes and native myocytes after adenoviral-transfer of the HERG-1a N-Terminal Domain fragment. Due to the different temperature dependence of kinetics of HERG-1a currents, these experiments in the permanently transfected-cell line are desirable for the direct comparison between kinetics of HERG-1a and native I_{Kr}. Identical ionic and temperature recording conditions are used for the cell-line and native myocyte determinations. In this way, a direct comparison of the deactivation kinetics of HERG-1a, native I_{Kr} and native I_{Kr} in the presence of the HERG 1a N-terminal domain is obtained.

The results of the experiments are found to indicate that transfer of the HERG1a N-Terminal Domain to native myocytes results in slower kinetics of deactivation of cardiac I_{Kr}. Deactivation kinetics measured for cells expressing the HERG-1a N-terminal domain and I_{Kr} are found to have deactivation kinetics that are the same as those of HERG-1a channels measured in the human cell line. Deactivation of I_{Kr} in wild-type myocytes and in cells infected with the negative control (HERG 1a Y43A domain) are found to have similar deactivation kinetics. Confocal microscopy localizes the HERG-1a N-Terminal Domain to the T-tubules (consistent with the localization of ERG-1b subunits to these membrane structures). Intact channels bearing Y43A are found to accelerate the kinetics of HERG in heart, suppress I_{Kr} and prolong the single cell action potential.

The experiments thus establish that the interaction of the introduced HERG-1a N-Terminal Domain with native ERG-1b subunits will convert ERG-1b channels or heteromeric ERG-1a/ERG-1b channels in vivo into channels with similar slow deactivation kinetics as in HERG-1a homomeric channels, thus explaining the mechanism that accounts for the relatively faster kinetics of I_{Kr}, relative to HERG1a deactivation kinetics.

The transfer of the HERG-1a N-Terminal Domain to native cells (via the anticipated slowing of I_{Kr} deactivation) increases the magnitude and duration of the resurgent I_{Kr} current and in turn shortens the cardiac action potential.

The experiments proposed above with the HERG1a N-terminal domain are necessary prior to measuring action potentials in cells expressing the HERG1a N-terminal domain.

Thus, the HERG-1a N-Terminal Domain represents a specific mechanism for correcting prolonged action potentials as both a specific mechanism for targeting unaffected HERG-1b channels that exist in HERG-1a subunit-specific LQTS, and correcting function of HERG channels bearing N-terminal region LQTS mutations, and a more general mechanism for shortening the QT interval by interacting with normal I_{Kr}. The present invention therefore additionally relates to the use of HERG-1a N-terminal regions, such as the HERG-1a N-Terminal Domain to remedy cardiac dysfunction (see, Sasano, T. et al. (2006) “Molecular Ablation Of Ventricular Tachycardia After Myocardial Infarction,” Nature Med. 12:1256-1258)).

### EXAMPLE 8

### The N-Terminal Domain of HERG-1a Interacts with Heteromeric Channels Comprised of HERG-1a and HERG-1b Subunits

The N-Terminal Domain of HERG-1a has been found to interact with heteromeric channels comprised of HERG-1a and HERG-1b subunits. Polynucleotides encoding the HERG-1a and HERG-1b proteins were expressed in Xenopus oocytes, and the HERG currents were measured with a two-electrode voltage-clamp. The results are shown in FIG. 13 for HERG-1a and HERG-1b (Panel A), and for HERG-1a and HERG-1b and HERG N-Terminal Domain (Panel B). HERG channels formed from co-expression of HERG-1a and HERG-1b (FIG. 13, Panel A, Panel C) had intermediated deactivation kinetics that were faster than those of HERG-1a (FIG. 7, Panel A) and slower than those of HERG-1b (FIG. 11).

In contrast, HERG channels formed from co-expression of HERG-1a, HERG-1b and the HERG1a N-Terminal Domain had deactivation kinetics that were significantly slower (FIG. 13, Panel B, Panel C) than those measured for HERG-1a and HERG-1b. Thus, the HERG-1a N-Terminal Domain slowed the deactivation kinetics (increased deactivation time constant) in heteromeric HERG-1a/HERG-1b channels (FIG. 13, Panel B, arrow).

These results indicate that, just as the HERG-1a N-Terminal Domain fragment is a specific, functional probe of HERG-1b channels, the HERG-1a N-Terminal Domain fragment is a specific, functional probe of channels formed from co-expression of HERG-1a and HERG-1b subunits. Notably, the kinetics of deactivation in the presence of the HERG-1a N-Terminal Domain fragment were identical to that of HERG-1a channels (FIG. 7, Panel A). Since native cardiac I_{Kr} is composed of ERG-1a and ERG-1b subunits, these results establish the validity and feasibility of probing native I_{Kr} with the HERG1a N-terminal domain fragment. Transfer of the HERG-1a N-terminal domain fragment to native ventricular myocytes causes slowing of the deactivation kinetics of native I_{Kr}.

The results directly show that the HERG-1a N-Terminal Domain fragment is translated inXenopus oocytes. Experiments were carried out by expressing HERG-1a N-Terminal Domain-eCFP in Xenopus oocytes, purifying and separating proteins with SDS-PAGE and detecting proteins on Westerns blots. Using an anti-GFP antibody, a specific band was detected at 37 kD, which is the predicted molecular size for the HERG-1a N-Terminal Fragment-eCFP fusion protein. Bands were also detected at and just above 50 kD, but were deemed to be non-specific since they were also detected in uninjected control oocytes. As a positive control, the HERG-1a N_{del}-mCitrine channels were purified and detected (predicted size 100 kD) at the proper molecular weight. The detection of the HERG-1a N-Terminal Region confirms the functional electrophysiological and fluorescence microscopy data and establishes that the Domain was translated in oocytes. Significantly, the ability to detect the HERG1a N-Terminal Domain and HERG N_{Del}-Citrine permits methods and assays for interaction that rely upon co-immunoprecipitation.

### EXAMPLE 9

### ERG-1a and ERG-1b Subunits Form Heteromeric Channels

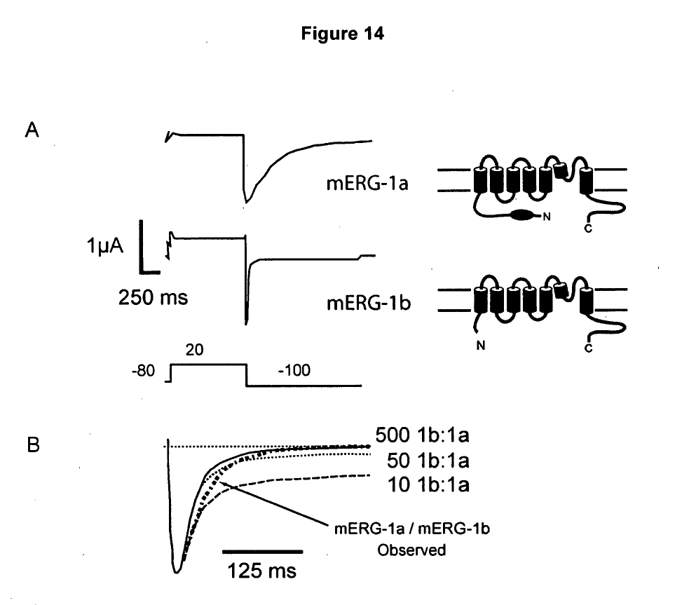

ERG-1a and ERG-1b subunits from mouse each can form functional homomeric channels when expressed alone (London, B. et al. (1997) “Two Isoforms Of The Mouse Ether-A-Go-Go-Related Gene Co-Assemble To Form Channels With Properties Similar To The Rapidly Activating Component Of The Cardiac Delayed Rectifier K+ Current,” Circ. Res. 81(5):870-878). mERG1a homomeric channels had properties identical to those of HERG-1a channels. In contrast, mERG-1b channels had closing kinetics (seen at -100 m V) that were approximately 10-fold faster than those for ERG-1a channels (FIG. 14, Panel A) (London, B. et al. (1997) “Two Isoforms Of The Mouse Ether-A-Go-Go-Related Gene Co-Assemble To Form Channels With Properties Similar To The Rapidly Activating Component Of The Cardiac Delayed Rectifier K+ Current,” Circ. Res. 81(5):870-878), consistent with a channel lacking the N-terminal region of ERG-1a (26,36,42,54). The difference in closing kinetics has been used to show that ERG-1a and ERG-1b formed heteromeric channels. mERG-1a and mERG-1b RNA at equal amounts were co-coexpressed and the inward tail current was measured (FIG. 14, Panel B, thick dotted trace). The measured current could not be explained by the simple summation of the tail currents from the homomeric current traces, nor could the measured current be explained by changing the weighting of ERG-1b current by 10- or 50- fold (FIG. 14, Panel B, thin dashed and dotted traces). Thus, the results indicate that the tail current measured after co-expression of mERG 1a and mERG 1b was not due to two populations of homomeric channels, but instead was due to heteromeric ERG 1a/ERG 1b channels with new properties.

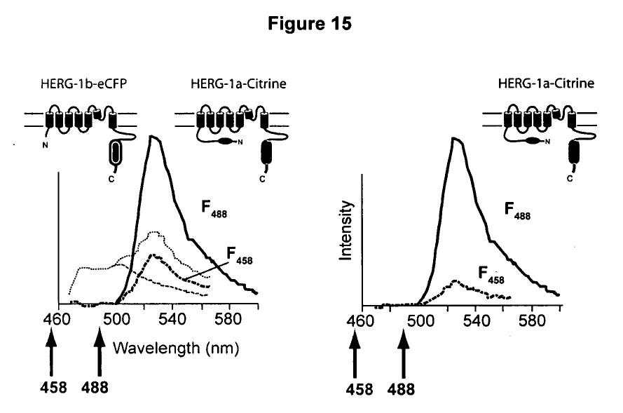

The subunit interactions between human ERG 1a and ERG 1b isoforms have been investigated using fluorescence resonance energy transfer (FRET). FRET occurs when fluorophores with overlapping emission and excitation spectra have a proper orientation are within approximately 100 A (Lakowitz, J. R. (1999) PRINCIPLES OF FLUORESCENT SPECTROSCOPY, Plennum Press, New York). Thus, FRET is a measure of physical proximity, and is an advance from inferring proximity from functional measurements (as in FIG. 14). The method used for measuring and analyzing FRET is described in detail in (Trudeau, M. C. et al. (2004) “Dynamics Of Ca²⁺-Calmodulin-Dependent Inhibition Of Rod Cyclicnucleotide-Gated Channels Measured By Patch-Clamp Fluorometry,” J. Gen. Physiol.124:211-223; Zheng, J. et al. (2002) “Rod-Cyclic Nucleotide Gated Channels Have A Stoichiometry Of Three CNGA1 Subunits And One CNGB1 Subunit,” Neuron. 36-891-896). Briefly, emission spectra was measured from oocytes expressing ion channel subunits fused to the fluorescent proteins enhanced Cyan Fluorescent Protein (eCFP) and Citrine (an improved Yellow Fluorescent Protein). eCFP and Citrine are FRET pairs, in which, following excitation of the donor, energy is transferred from the donor (eCFP) to the acceptor (Citrine). Emission spectra were measured by laser scanning confocal microscopy, from oocytes co-expressing HERG 1b-eCFP and HERG 1a-Citrine (FIG. 15, left) or HERG-1a-Citrine alone (FIG. 15, right). FRET was measured as stimulated emission of the acceptor (Citrine) by the donor (eCFP). In the experiment with HERG-1b-eCFP and HERG-1a-Citrine (FIG. 15, left), the ratio (Ratio A) of the spectra after excitation by the 458 laser line (F458, thick dashed) compared to the spectra after excitation with the 488 laser (F 488, solid) is larger than the ratio (Ratio A₀) of the F₄₅₈ spectra (thick dashed) to the F₄₈₈ spectra (solid) in the control experiment with HERG I a-Citrine alone (FIG. 15, right). The significant difference (Ratio A-Ratio A₀ value=0.18±0.03, n=8) showed FRET between HERG-1a and HERG-1b subunits. A similar amount of FRET was also detected between HERG-1a-eCFP and HERG-1a-Citrine. The FRET measured here is similar in magnitude to that seen with heteromeric cyclic nucleotide-gated (CNG) channels that were labeled with fluorescent proteins (Trudeau, M. C. et al. (2004) “Dynamics Of Ca²⁺-Calmodulin-Dependent Inhibition Of Rod Cyclicnucleotide-Gated Channels Measured By Patch-Clamp Fluorometry,” J. Gen. Physiol. 124:211-223; Zheng, J. et al. (2002) “Rod-Cyclic Nucleotide Gated Channels Have A Stoichiometry Of Three CNGA1 Subunits And One CNGB1 Subunit,” Neuron. 36-891-896). These results show that HERG-1a and HERG-1b subunits are in close proximity in the cell membrane and form heteromeric HERG-1a/HERG-1b channels. These results also establish the feasibility of using FRET to investigate subunit stoichiometry of HERG-1a/HERG-1b channels.

### EXAMPLE 10

### The C-Terminal Domain of HERG-1a

The C-terminal region of HERG-1a is a large domain that includes a putative cyclic nucleotide-binding domain (CNBD), a “C-linker” region connecting the CNBD to the S6 region, and a region distal to the CNBD (Warmke, J. W. et al. (1994) “A Family Of Potassium Channel Genes Related To Eag In Drosophila And Mammals,” Proc. Natl. Acad. Sci. (USA) 91(8):3438-3442). Deletion mutations of regions downstream from the CNBD in HERG resulted in channels with fast deactivation kinetics, similar to the kinetics in channels with N-terminal deletions (Aydar, E. et al. (2001) “Functional Characterization Of The C-Terminus Of The Human Ether-A-Go-Go-Relatedgene K(+)Channel(HERG),” J. Physiol. 534:1-14). Double deletions of the N— and C-terminal regions result in HERG channels with kinetics of deactivation that are not simply additive, suggesting that the mechanism for modulation of deactivation may involve a physical interaction between the N— and C-terminal regions (Aydar, E. et al. (2001) “Functional Characterization Of The C-Terminus Of The Human Ether-A-Go-Go-Relatedgene K(+)Channel(HERG),” J. Physiol. 534:1-14). Interactions between the N— and C-terminal regions have been found in other K⁺ channels (Kuo, A. et al. (2003) “Crystal Structure of the Potassium Channel KirBac1.1 in the Closed State,” Science 300:1922-1926; Schulteis, C. T et al. (1996) “Intersubunit Interaction Between Amino- And Carboxyl-Terminal Cysteine Residues In Tetrameric Shaker K⁺ Channels,” Biochem. 35:12133-12140) and in CNG channels, which, like BERG containa CNBD in the C-terminal region (Gordon, S. E. et al. (1997) “Direct Interaction Between Amino- And Carboxyl-Terminal domains Of Cyclic Nucleotide-Gated Channels,” Neuron 19:431-441; Trudeau, M. C. et al. (2004) “Dynamics Of Ca²⁺-Calmodulin-Dependent Inhibition Of Rod Cyclicnucleotide-Gated Channels Measured By Patch-Clamp Fluorometry,” J. Gen. Physiol. 124:211-223; Varnum, M. D. et al. (1997) “Interdomain Interactions Underlying Activation Of Cyclic Nucleotide-Gatedchannels,” Science 278:110-113; Zheng, J. et al. (2003) “Disruption Of An Intersubunit Interaction Underlies Ca²⁺-Calmodulin Modulation Of Cyclic Nucleotide-Gated Channels,” J. Neurosci. 23:8167-8175), indicating that such interactions are a common feature in these ion channels.

As discussed above, HERG-1b plays a role in establishing I_{Kr} (Lees-Miller, J. P. et al. (2003) “Selective Knockout of Mouse ERG1B Potassium Channel Eliminates I(_{Kr}) In Adult Ventricular Myocytes And Elicits Episodes Of Abrupt Sinus Bradycardia,” Mol. Cell. Biol. 23:1856-18562; London, B. et al. (1997) “Two Isoforms Of The Mouse Ether-A-Go-Go-Related Gene Coassemble To Form Channels With Properties Similar To The Rapidly Activating Component Of The Cardiac Delayed Rectifier K+ Current,” Circ. Res. 81(5):870-878). Ancillary subunits are also thought to play a key role in forming I_{Kr} (Abbott, G. W. et al. (1999) “MiRP1 Forms I_{Kr} Potassium Channels With HERG And Is Associated With Cardiac Arrhythmia,” Cell 97:175-187).

Isoforms 3 and 4 of HERG-1a contain C-terminal deletions. HERG-1a isoform 3 is abundantly present in heart cells (Kupershmidt, S. et al. (1998) “A K+ Channel Splice Variant Common In Human Heart Lacks A C-Terminal Domain Required For Expression Of Rapidly Activating Delayed Rectifier Current,” J. Biol. Chem. 273(42):27231-27235). Analogous to the role of the N-terminal deletion of HERG-1b as an in vivo regulator of HERG-1a deactivation kinetics, isoforms 3 and 4 of HERG-1a also function in vivo to accelerate HERG-1a deactivation kinetics. Accordingly, the C-terminal domain of HERG-1a is capable of restoring slow deactivation kinetics to channels formed from HERG-1a isoform 3 or isoform 4. The C-terminal domain of HERG-1a can therefore be employed to assay for HERG-1a isoform 3 or isoform 4 function and dysfunction.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method for decreasing the deactivation kinetics of the I_{Kr} current of a mammalian cardiac cell which comprises providing to said cell a compound that specifically antagonizes a function of ERG-1b.

2. The method of claim 1, wherein said cell is a human cell, and said ERG-1b is HERG-1b.

3. The method of claim 2, wherein said compound comprises a polypeptide or peptide fragment of the Amino Terminal Domain of HERG-1a.

4. The method of claim 2, wherein said compound is the Amino Terminal Domain of HERG-1a.

5. The method of any of claims 3 or 4, wherein said provision of said compound to said cell is accomplished by providing to said cell a polynucleotide encoding said compound under conditions sufficient to cause expression of said polynucleotide.

6. A method for decreasing the deactivation kinetics of the I_{Kr} current of a mammalian cardiac cell which comprises providing to said cell a compound that specifically antagonizes a function of an ERG-1a molecule that comprises a mutation relative to the amino acid sequence of the wild-type ERG-1a protein.

7. The method of claim 6, wherein said cell is a human cell, and said ERG-1a is HERG-1a.

8. The method of claim 6, wherein said ERG-1a comprises a mutation in the PAS domain.

9. The method of claim 8, wherein the mutation in the PAS domain is at position 31 and/or 43.

10. The method of claim 6, wherein said compound comprises a polypeptide or peptide fragment of the Amino Terminal Domain of HERG-1a.

11. The method of claim 6, wherein said compound is the Amino Terminal Domain of HERG-1a.

12. The method of any of claims 10 or 11, wherein said provision of said compound to said cell is accomplished by providing to said cell a polynucleotide encoding said compound under conditions sufficient to cause expression of said polynucleotide.

13. A method for increasing the deactivation kinetics of the I_{Kr} current of a mammalian cardiac cell which comprises providing to said cell a compound that specifically antagonizes a function of ERG-1a.

14. The method of claim 13, wherein said cell is a human cell, and said ERG-1a is HERG-1a.

15. The method of claim 13, wherein said compound comprises a polypeptide or peptide fragment of HERG-1b or of HERG-1a isoform 3 or 4.

16. The method of any of claims 14 or 15, wherein said provision of said compound to said cell is accomplished by providing to said cell a polynucleotide encoding said compound under conditions sufficient to cause expression of said polynucleotide.

17. A method for evaluating ERG channel composition or function in a sample membrane containing said channel, wherein said method comprises the steps of:
(A) providing to said sample membrane a compound that specifically antagonizes a function of an ERG-1 subunit of an ERG channel; and
(B) determining the effect of said compound on the deactivation kinetics of the I_{Kr} current of said sample membrane relative to the deactivation kinetics of the I_{Kr} current of a reference membrane in the presence of said compound;
wherein a difference in the effect of said compound on the I_{Kr} current deactivation kinetics of said sample membrane relative to said reference membrane indicates that said sample membrane exhibits abnormal ERG channel composition or function.

18. The method of claim 17, wherein said sample membrane is the membrane of a cell.

19. The method of claim 17, wherein said sample membrane is an in vitro membrane.

20. The method of claim 17, wherein said ERG-1 subunit is ERG-1a.

21. The method of claim 17, wherein said ERG-1 subunit is ERG-1b.

22. The method of claim 21, wherein said compound comprises a polypeptide or peptide fragment of the Amino Terminal Domain of HERG-1a.

23. A method for determining whether an agent affects ERG channel function, wherein said method comprises the steps of:
(A) providing said agent to a membrane that comprises an ERG channel; and
(B) determining whether said agent alters the deactivation kinetics of the I_{Kr} current of said membrane;
wherein a difference in the I_{Kr} current deactivation kinetics of said membrane in the presence of said agent relative to the I_{Kr} current deactivation kinetics of said membrane in the absence of said agent indicates that said agent affects ERG channel function.

24. The method of claim 23, wherein said sample membrane is the membrane of a cell.

25. The method of claim 23, wherein said sample membrane is an in vitro membrane.

26. The method of claim 23, wherein said ERG-1 subunit is ERG-1a.

27. The method of claim 23, wherein said ERG-1 subunit is ERG-1b.

28. The method of claim 23, wherein said agent is an antiarrhythmic agent.

29. The method of claim 23, wherein said agent is a non-antiarrhythmic agent.

30. The method of claim 23, wherein said agent is an antineoplastic agent.


SEQUENCE LISTING (21 sequences)

SEQ ID NO: 1 - protein, 1159 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 2 - DNA, 3480 bp, Homo sapiens
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttag

SEQ ID NO: 3 - protein, 819 aa, Homo sapiens
MAAPAGKASRTGALRPRAQKGRVRRAVRISSLVAQEVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 4 - protein, 888 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGMGWGAGTGLEMPSAASRGASLLNMQSLGLWTWDCLQGHWAPLIHLNSGPPSGAMERSPTWGEAAELWGSHILLPFRIRHKQTLFASLK

SEQ ID NO: 5 - protein, 823 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGMGWGAGTGLEMPSAASRGASLLNMQSLGLWTWDCLQGHWAPLIHLNSGPPSGAMERSPTWGEAAELWGSHILLPFRIRHKQTLFASLK

SEQ ID NO: 6 - protein, 135 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEK

SEQ ID NO: 7 - protein, 16 aa, Homo sapiens
MPVRRGHVAPQNTFLD

SEQ ID NO: 8 - protein, 35 aa, Homo sapiens
GISSLFSSLKVVRLLRLGRVARKLDHYIEYGAAVL

SEQ ID NO: 9 - protein, 35 aa, Homo sapiens
GSEELIGLLKTARLLRLVRVARKLDRYSEYGAAVL

SEQ ID NO: 10 - protein, 11 aa, Homo sapiens
YGRKKRRQRRR

SEQ ID NO: 11 - protein, 17 aa, Artificial
  His 6 leader fused to SEQ ID NO10
HHHHHHYGRKKRRQRRR

SEQ ID NO: 12 - protein, 6 aa, Artificial
  His 6 Leader Sequence
HHHHHH

SEQ ID NO: 13 - DNA, 405 bp, Homo sapiens
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaagtggtgatggagaag

SEQ ID NO: 14 - DNA, 720 bp, Aequorea victoria
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 15 - protein, 239 aa, Aequorea victoria
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAAGITLGMDELYK

SEQ ID NO: 16 - protein, 239 aa, Aequorea victoria
MVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLGMDELYK

SEQ ID NO: 17 - DNA, 720 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 18 - DNA, 2418 bp, Homo sapiens
atggaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagt

SEQ ID NO: 19 - protein, 805 aa, Homo sapiens
MREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 20 - protein, 21 aa, Homo sapiens
LSRHSKGLQILGQTLKASMRE

SEQ ID NO: 21 - protein, 21 aa, Homo sapiens
RYSEYGAAVLFLLMCTFALIA